(19) United States
(12) Patent Application Publication
(10) Pub. No.: US 2025/0339688 A1
(43) Pub. Date: Nov. 6, 2025

(54) TREATMENT OF TISSUE USING ELECTRICAL STIMULATION

(71) Applicant: SOMNIAL INC., Stony Brook, NY (US)

(72) Inventors: William HANES, East Setauket, NY (US); Adam WILLIAMSON, Norrköping (SE); Mary DONAHUE, Norrköping (SE); Malin Silverå EJNEBY, Linköping (SE); Florian MISSEY, Marseille (FR)

(21) Appl. No.: 19/272,733

(22) Filed: Jul. 17, 2025

Related U.S. Application Data
(63) Continuation of application No. PCT/US2024/012039, filed on Jan. 18, 2024.
(60) Provisional application No. 63/439,635, filed on Jan. 18, 2023.

(51) Int. Cl. A61N 1/36; A61N 1/04

(52) U.S. Cl. CPC A61N 1/3606; A61N 1/0476; A61N 1/36171

(57) ABSTRACT

An apparatus for electrical stimulation of tissues includes four electrodes that include a first pair of electrodes and a second pair of electrodes. The first and second pair of electrodes are each configured to contact a person and to convey AC current between the respective pair of electrodes through tissue of the person. The four electrodes are positioned approximately in the shape of a square where the first pair of electrodes and the second pair of electrodes are each positioned at opposite vertices of the square. The first pair of electrodes and the second pair of electrodes are configured to be positioned such that their AC currents are simultaneously conveyed through a target tissue of the person and are configured to stimulate the target tissue.

### CROSS-REFERENCE TO RELATED APPLICATIONS

The present application is a Continuation of International Patent Application No. PCT/US2024/012039, filed on Jan. 18, 2024, which claims the benefit of and priority to U.S. Provisional Patent Application No. 63/439,635, filed on Jan. 18, 2023. The entire contents of each of the foregoing applications are hereby incorporated by reference herein.

### TECHNICAL FIELD

The present disclosure relates generally to electrical stimulation of tissue and, more particularly, to treating diseases by electrical stimulation.

### BACKGROUND

Disorders associated with the airway reduce airway volume, restrict airflow, and prevent adequate respiration. These disorders may occur in diseases such as Obstructive Sleep Apnea (OSA), but can also occur in other circumstances, such as when patients are under sedation. Airflow through a patient's airway may be reduced due to partial blockage by the tongue. OSA may manifest with repeated collapse of the airway during sleep due to relaxation of the upper airway dilator muscles. In patients with OSA, the tongue muscle loses tone and relaxes, causing the tongue to slide backward in the mouth and narrow the pharynx. OSA contributes to upper airway obstruction, loss of breathing control, and a loss of oxygenation and gas exchange, which can lead to intermittent hypoxia. Existing treatments for OSA focus on opening the airway and increasing airflow or require surgical implants. Continuous positive airway pressure (CPAP) is a therapy which forces airflow through a mask while the patient sleeps. CPAP and surgical implants are invasive and uncomfortable treatments that leave much to be desired. OSA is but one example, among many, of diseases whose treatments have great room for improvement.

### SUMMARY

The present disclosure relates to the use of electrical current to stimulate tissue.

In accordance with aspects of the present disclosure, an apparatus for electrical stimulation of tissue includes: four electrodes that include: a first pair of electrodes configured to contact a person and to convey a first AC current between the first pair of electrodes through tissue of the person, and a second pair of electrodes configured to contact the person and to convey a second AC current between the second pair of electrodes and through tissue of the person, where the four electrodes are positioned approximately in a shape of a square, where the first pair of electrodes and the second pair of electrodes are each positioned at opposite vertices of the square, and where the first pair of electrodes and the second pair of electrodes are configured to be positioned on the person such that the first AC current and the second AC current are simultaneously conveyed through a target tissue of the person, and the first AC current and the second AC current configured to stimulate the target tissue.

In various embodiments of the apparatus, the first AC current has a first frequency and the second AC current has a second frequency, where the first frequency and the second frequency are both at least 5000 Hz.

In various embodiments of the apparatus, current conveyed through the target tissue has a frequency different from the first frequency and different from the second frequency.

In various embodiments of the apparatus, the current conveyed through the target tissue has a frequency equivalent to a frequency difference between the first frequency and the second frequency.

In various embodiments of the apparatus, the frequency difference is approximately 50 Hz.

In various embodiments of the apparatus, the first frequency is approximately 5000 Hz and the second frequency is approximately 5050 Hz.

In various embodiments of the apparatus, the first frequency is approximately 6000 Hz and the second frequency is approximately 6050 Hz.

In various embodiments of the apparatus, edges of the square are approximately 2 cm.

In various embodiments of the apparatus, the apparatus further includes a patch configured to adhere to skin of the person, where the patch includes the first pair of electrodes and the second pair of electrodes.

In various embodiments of the apparatus, the apparatus further includes: a second set of four electrodes that includes: a third pair of electrodes configured to contact the person and to convey a third AC current between the third pair of electrodes and through tissue of the person, and a fourth pair of electrodes configured to contact the person and to convey a fourth AC current between the fourth pair of electrodes and through tissue of the person, where the four electrodes of the second set are positioned approximately in a shape of a second square, where the third pair of electrodes and the fourth pair of electrodes are each positioned at opposite vertices of the second square.

In various embodiments of the apparatus, the first AC current has a first frequency, the second AC current has a second frequency, the third AC current has a third frequency, and the fourth AC current has a fourth frequency, wherein the third frequency and the fourth frequency are both at least 5000 Hz.

In various embodiments of the apparatus, the first frequency is approximately 5000 Hz, the second frequency is approximately 5050 Hz, third frequency is approximately 6000 Hz, and the fourth frequency is approximately 6050 Hz.

In various embodiments of the apparatus, edges of the second square are approximately 2 cm.

In various embodiments of the apparatus, the third pair of electrodes and the fourth pair of electrodes are configured to be positioned on the person such that the third AC current and the fourth AC current are simultaneously conveyed through the target tissue of the person, and the third AC current and the fourth AC current are configured to stimulate the target tissue.

In various embodiments of the apparatus, the target tissue includes a hypoglossal nerve of the person.

In various embodiments of the apparatus, the apparatus further includes a patch configured to adhere to skin of the person, where the patch includes the first pair of electrodes, the second pair of electrodes, the third pair of electrodes, and the fourth pair of electrodes.

In various embodiments of the apparatus, the patch is configured to be affixed to skin of the person under a mandible of the person.

In various embodiments of the apparatus, the patch has a shape that tracks a jawline of the person.

Further aspects and embodiments include the Examples set forth in the Examples section below.

Further details and exemplary aspects of the present disclosure are described in more detail below with reference to the appended figures. Any of the aspects of the present disclosure may be combined with other aspects without departing from the scope of the present disclosure.

### BRIEF DESCRIPTION OF THE DRAWINGS

A better understanding of the features and advantages of the disclosed technology will be obtained by reference to the following detailed description that sets forth illustrative aspects, in which the principles of the technology are utilized, and the accompanying drawings of which:

FIG. 1 is a block diagram of exemplary components of a system for electrically stimulating tissue, in accordance with aspects of the disclosure;

FIG. 2 is a diagram of exemplary electrical outputs of two electrical stimulators, in accordance with aspects of the disclosure;

FIG. 3 is another diagram of exemplary electrical outputs of two electrical stimulators, in accordance with aspects of the disclosure;

FIG. 4 is a diagram of exemplary electrical output frequencies of two electrical stimulators, in accordance with aspects of the disclosure;

FIG. 5 is a diagram of exemplary electrode configurations, in accordance with aspects of the disclosure;

FIG. 6 is a diagram of exemplary electrode array configurations, in accordance with aspects of the disclosure;

FIG. 7 is a diagram of an exemplary operation for controlling electrical outputs of electrodes, in accordance with aspects of the present disclosure;

FIG. 8 is a diagram of an exemplary curved housing having electrodes, in accordance with aspects of the disclosure;

FIG. 9 is a diagram of exemplary charging pins on the curved housing of FIG. 8, in accordance with aspects of the present disclosure;

FIG. 10 is a diagram of an exemplary docking device, in accordance with aspects of the present disclosure;

FIG. 11 is a diagram of exemplary components of an apparatus, in accordance with aspects of the present disclosure;

FIG. 12 is a diagram of the tongue and related tissue;

FIG. 13 is a diagram of an exemplary apparatus placed on a person below the mandible, in accordance with aspects of the present disclosure;

FIGS. 14 and 15 are diagrams of an exemplary apparatus placed inside the oral cavity, in accordance with aspects of the disclosure;

FIG. 16 is a diagram of tongue movement, in accordance with aspects of the disclosure;

FIG. 17 is a diagram of exemplary patches placed on a person, in accordance with aspects of the disclosure;

FIG. 18 is a diagram of another exemplary patch placed on a person below the mandible, in accordance with aspects of the disclosure;

FIG. 19 is a diagram of exemplary placement of electrodes on a person, in accordance with aspects of the disclosure;

FIG. 20 is a diagram of another exemplary placement of electrodes on a person, in accordance with aspects of the disclosure;

FIG. 21 is a block diagram of an operation for electrical stimulation of tissue, in accordance with aspects of the disclosure;

FIG. 22 is a flow diagram of an exemplary operation for electrically stimulating a hypoglossal nerve, in accordance with aspects of the disclosure;

FIG. 23 is a flow diagram of an exemplary operation for electrically stimulating a neuron, in accordance with aspects of the disclosure;

FIG. 24 is a diagram of an exemplary operation of providing power, in accordance with aspects of the disclosure;

FIG. 25 is a diagram of an exemplary patch with arrangement of four electrodes, in accordance with aspects of the disclosure;

FIG. 26 is a diagram of exemplary electric fields generated by the electrodes of FIG. 25, in accordance with aspects of the disclosure;

FIG. 27 is a diagram of exemplary voltage frequencies applied to the electrodes of FIG. 25, in accordance with aspects of the disclosure;

FIG. 28 is a diagram of exemplary voltage frequencies applied to two patches, in accordance with aspects of the disclosure;

FIG. 29 is a diagram of exemplary timing of when the voltages of FIG. 28 are applied or not applied, in accordance with aspects of the disclosure;

FIG. 30 is a diagram of an exemplary pair of patches, in accordance with aspects of the disclosure;

FIG. 31 is a diagram of an exemplary patch showing top and bottom surfaces, in accordance with aspects of the disclosure;

FIG. 32 is a diagram of exemplary patches with one patch having an exemplary connector, in accordance with aspects of the disclosure;

FIG. 33 is a diagram of an exemplary curved housing having four electrodes on each side, in accordance with aspects of the disclosure;

FIG. 34 is a diagram of the curved housing with electrodes of FIG. 33 and an exemplary charger, in accordance with aspects of the disclosure;

FIG. 35 is a schematic diagram of an exemplary stimulation device with a connection to a cable, in accordance with aspects of the disclosure;

FIG. 36 is a schematic diagram of another exemplary stimulation device with a sensor on the cable, in accordance with aspects of the disclosure;

FIG. 37 is a schematic diagram of an exemplary stimulation device with a magnetic connection to a cable, in accordance with aspects of the disclosure;

FIG. 38 is a schematic diagram of an exemplary stimulation system having a stimulation device and a control device connected by a cable, in accordance with aspects of the disclosure;

FIG. 39 is a graph showing clinical Apnea Hypopnea Index (AHI) data for patients when stimulated and when not stimulated by a stimulation device;.

FIG. 40 is a diagram of an exemplary placement of four electrode pairs on a person, in accordance with aspects of the disclosure;

FIG. 41 is a diagram of another exemplary placement of four electrode pairs on a person, in accordance with aspects of the disclosure; and

FIG. 42 is a diagram of yet another exemplary placement of four electrode pairs on a person, in accordance with aspects of the disclosure;

### DETAILED DESCRIPTION

The present disclosure relates to the use of electrical current to stimulate tissue. Aspects of the present disclosure use electrical stimulation to treat diseases (such as obstructive sleep apnea and other diseases) in a non-invasive manner that does not involve surgery or surgical implants. As described below, current is conveyed through tissue between electrodes, and multiple currents from multiple electrodes are configured electrically and spatially to stimulate or provide treatment of target tissue. As used herein, tissue is “stimulated” when current flowing through the tissue causes a muscle movement, such as a muscle contraction, and/or causes nerve depolarization. When current flowing through tissue does not cause any muscle movement or nerve depolarization, the tissue would not be stimulated. When the target tissue includes motor neurons, for example, electrical stimulation of the motor neurons may cause associated muscle fibers to react in a desired manner and, thereby, effectuate treatment of a disease.

As used herein, the term “exemplary” is intended to mean “example” and is not intended to mean “preferred.” Unless indicated otherwise, the terms “apparatus” and “system” may be used interchangeably, and each term is not intended to mean or imply any particular structure. For example, apparatuses or systems disclosed herein may be embodied in various structures, such as embodied in a single housing or embodied in more than one housing.

In the following detailed description, specific details are set forth to provide an understanding of aspects of the disclosure and to provide various examples. It will be understood by those skilled in the art that aspects of the disclosure may be practiced without using the exact details described herein and may be practiced in manners not specifically described herein. In various instances, well-known methods, procedures, and/or components are not described in detail so as to not obscure the present disclosure. Unless the context indicates otherwise, any or all of the aspects, embodiments, and examples detailed herein may be used in conjunction with any or all of the other aspects or embodiments detailed herein.

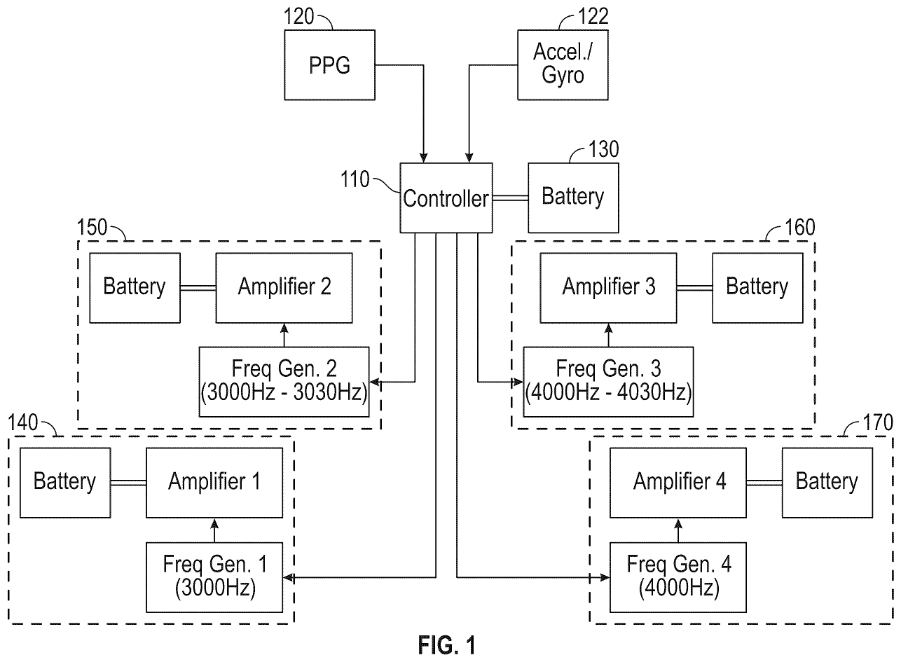

FIG. 1 shows a block diagram of exemplary components of a system or apparatus for electrically stimulating tissue. For convenience, the term “system” will be used to describe FIG. 1, but it is intended that any description using the term “system” shall be treated as if the same description used the term “apparatus” as well. In the illustrated embodiment of FIG. 1, the components include a controller 110, various sensors 120, 122, a battery 130 for the controller 110, and multiple electrical stimulators 140-170 that generate electrical stimulation. The controller 110 may be or include any computational device, including microcontrollers, microprocessors, digital signal processors, central processing units (CPUs), graphics processing units (GPUs), application specific integrated circuits (ASICs), programmable logic devices (PLDs), and/or field-programmable gate arrays (FPGAs), among other computational devices. The controller 110 is powered by the battery 130, which may be a rechargeable battery or a non-rechargeable battery. The controller 110 includes input and/or output (“I/O”) connections to the sensors 120, 122. The I/O connections may be analog I/O connection or digital I/O connections. The illustrated sensors include a photoplethysmography (PPG) sensor 120 and an acceleration/gyroscopic sensor 122. However, the illustrated sensors are merely examples, and the sensors connected to the controller 110 may include any type and any number of sensors supported by the controller 110.

The controller 110 includes I/O connections to the electrical stimulators 140-170. The I/O connections may be analog I/O connection or digital I/O connections. In the illustrated embodiment, each electrical stimulators 140-170 includes a battery, a frequency generator, and an amplifier. In embodiments, the frequency generator of each stimulator 140-170 may be a voltage controlled oscillator that is controlled by an analog I/O connection of the controller 110. The amplifier of each electrical stimulator 140-170 may be any type of amplifier, which persons skilled in the art will recognize. The battery of each electrical stimulator 140-170 may be a rechargeable battery or a non-rechargeable battery. Each electrical stimulator 140-170 also includes electrodes (not shown) that can be arranged in various ways, and such electrodes and arrangements will be described later herein.

In accordance with aspects of the present disclosure, the controller 110 can independently direct each of the electrical stimulators 140-170 to provide a desired electrical output, such as a desired voltage, desired current, and/or desired frequency, among other electrical outputs. For example, the controller 110 can independently direct each of the electrical stimulation blocks 140-170 to provide AC current of a desired frequency or within a desired frequency range, such as the exemplary frequencies/ranges shown in FIG. 1. Because each electrical stimulator 140-170 has a dedicated battery, the electrical output of each electrical stimulation 140-170 may be controlled more precisely by the controller 110. The effect of the electrical stimulators 140-170 may depend on the properties of their electrical outputs (e.g., frequencies) and/or the physical locations where their electrodes are placed on a person. Examples of such electrical properties and physical locations are described below.

In accordance with aspects of the present disclosure, the controller 110 may control the electrical stimulators 140-170 in various ways based on the outputs of the sensors 120, 122. For example, the controller 110 may direct one or more electrical stimulators 140-170 to provide electrical output based on the output(s) of one or both of the sensors 120, 122.

The system illustrated in FIG. 1 is exemplary, and variations are contemplated to be within the scope of the present disclosure. In embodiments, the number of electrical stimulators may be more than four or less than four. In embodiments, the stimulation of the tissue can be tonic and constant or turned on and off periodically. When turned on and off, the stimulation may be regular, random, variable, or based upon an event. Various events may be configured to trigger activation of stimulation, including a breathing event, an apnea event, and/or a user-defined event such as the passage of time. In embodiments, the primary trigger may be a breathing event. The breathing event may be detection that a breath is taken or may be defined by an algorithm. An algorithmic event may include variety of variables, such as oxygenation levels, breathing rates, and exhaustion through overuse of the muscles. In embodiments, the sensors may be configured to detect breathing events, including chest-based accelerometers, sound meters, electrical activity sensors, and pressure transducers.

For example, in embodiments, a chest-based accelerometer may measure the movements of the body. In embodiments, a sound meter may detect breathing sounds. In embodiments, recordings of the electrical activity of the phrenic nerve, which controls the diaphragm during breathing, may be analyzed to detect a breath. In embodiments, a pressure transducer implanted in the chest may sense the changes in pressure associated with a breathing event. In embodiments, breathing rates may be determined from plethysmography traces.

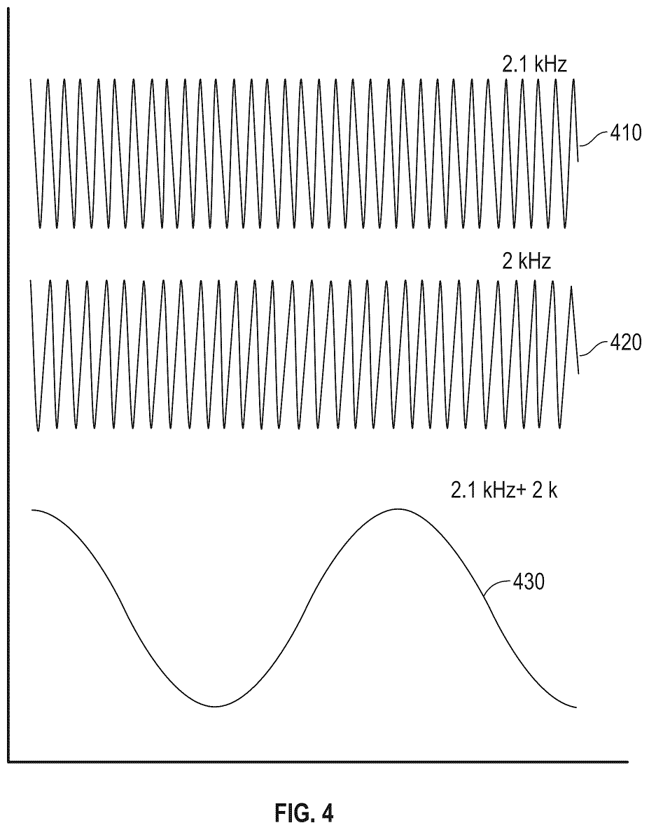

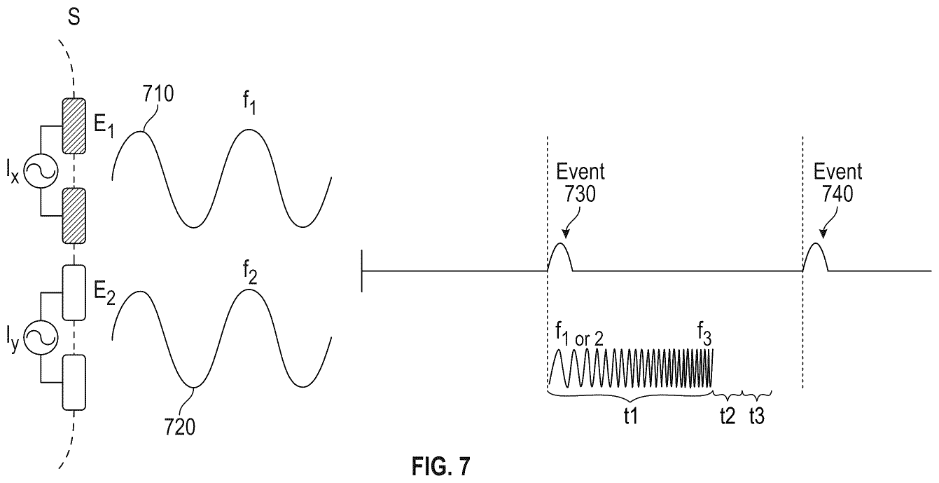

In embodiments, the stimulation of the tissue may be activated in response to an algorithm detecting breathing events in real time. In embodiments, the stimulation of the tissue may be activated based on an algorithm predicting breathing events, such as a moving average of previously measured breathing events. In embodiments, the tissue may be stimulated without detection of breathing events, such as regular periodic stimulations or semi-random stimulations. The frequency and current of stimulations may be adjustable and can modulate between periodic stimulations, or the frequency may sweep from an “Off” frequency to an “On” frequency, which will be described in more detail in connection with FIG. 4 and FIG. 7.

Persons skilled in the art will understand that certain components not shown in FIG. 1 may be included in the system of FIG. 1, such as memory, data and/or machine-readable instructions stored in the memory, and communication circuitry, among other components. Such and other variations are contemplated to be within the scope of the present disclosure.

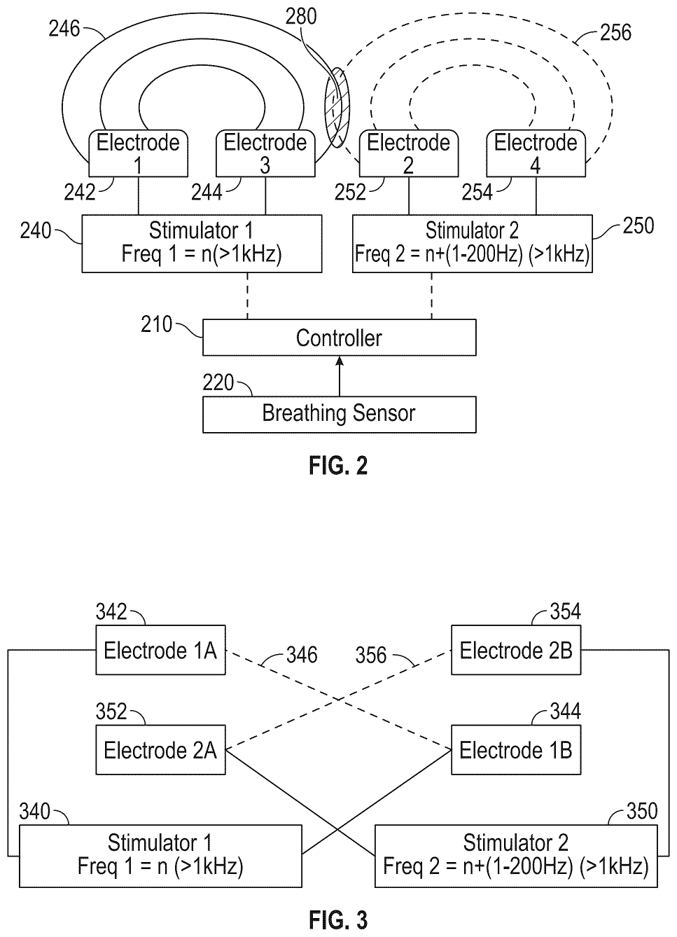

FIG. 2 is a diagram of exemplary electrical outputs of two electrical stimulators. The illustrated system includes a controller 210, a breathing sensor 220, a first electrical stimulator 240 having electrodes 242, 244, and a second electrical stimulator 250 having electrodes 252, 254. The electrical stimulators 240, 250 may include the components of the electrical stimulators shown in FIG. 1. The controller 210 may control the two electrical stimulators 240, 250 to provide electrical outputs having different frequencies, such as the frequencies shown in FIG. 2. For example, both electrical stimulators 240, 250 may provide electrical outputs having frequencies greater than 1 kHz. One electrical stimulator (e.g., 250) may provide an electrical output frequency that is slightly higher, such as higher by 1 Hz-100 Hz, by 1 Hz-200 Hz, or by 1 Hz-1000 Hz, for example. The frequencies are exemplary, and other frequencies are contemplated to be within the scope of the present disclosure.

The electrodes of each electrical stimulator cause current to flow between them. The electrodes 242, 244 of the first electrical stimulator 240 may have corresponding current(s) 246 flow between them, and the electrodes 252, 254 of the second electrical stimulator 250 may have corresponding current(s) 256 flow between them. As shown in FIG. 2, the illustrated paths of the currents 246, 256 intersect at two locations. The illustrated paths are provided for explanatory purposes, and actual current paths may not have the illustrated shapes. In embodiments, the paths of the currents 246, 256 may intersect at one location or may intersect at more than two locations.

In accordance with aspects of the present disclosure, the currents 246, 256 flowing between the electrodes of the electrical stimulators 240, 250 may simultaneously flow through a target tissue 280 and may stimulate the target tissue 280. The effect of the currents 246, 256 in the target tissue 280 will be described in more detail in connection with FIG. 4. The controller 210 may control the timing and/or electrical properties of the currents 246, 256 (e.g., frequency, amplitude, etc.) to provide a desired effect on the target tissue 280. For example, in embodiments, if the target tissue 280 includes motor neurons, the effect of the currents 246, 256 may cause muscle fibers associated with the motor neurons to be activated, thereby causing muscle contraction in a person. In embodiments, the neurons of the target tissue 280 may include a hypoglossal nerve. If the breathing sensor 220 indicates reduction or stoppage of breathing, the controller 210 may control the electrical stimulators 240, 250 to stimulate the hypoglossal nerve of target tissue 280 to cause the tongue to move. Other types of target tissue and other effects of applying electrical outputs to target tissue are contemplated to be within the scope of the present disclosure.

FIG. 3 is another diagram of exemplary electrical outputs of two electrical stimulators. The first simulator 340 causes an electrical current 346 to flow between its electrodes 342, 344, and the second simulator 350 causes an electrical current 356 to flow between its electrodes 352, 354. The electrical stimulators 340, 350 may include the components of the electrical stimulators shown in FIG. 1. The electrodes 342, 344, 352, 354 are physically located on a person in locations that result in the currents 346, 356 crossing paths at a location. The illustrated current paths 346, 356 are shown for explanatory purposes, and actual current paths may have a different shape. In embodiments, the paths of the currents 346, 356 may intersect at more than one location. In embodiments, the currents 346, 356 cross paths at a target issue and stimulate or otherwise effect treatment of the target tissue. For example, the currents 346, 356 may have the frequencies shown in FIG. 3 and may provide treatment in the target tissue. The frequencies shown in FIG. 3 are exemplary, and other frequencies are contemplated to be within the scope of the present disclosure.

The systems described in connection with FIGS. 1-3 may be used to stimulate neurons and muscle fibers. As described above, tissue is “stimulated” when current flowing through the tissue causes a muscle movement, such as a muscle contraction, and/or causes nerve depolarization. When current flowing through tissue does not cause any muscle movement or nerve depolarization, the tissue would not be stimulated. Neurons and muscle fibers can be stimulated by electrical current at sub-kilohertz frequencies. At frequencies above 1 kHz, neurons may not respond and muscle fibers may not move.

At an electrical level, each electrode pair provides an electric field, and current(s) flowing between the electrodes of an electrode pair are based on the electric field. When multiple electrode pairs output electric fields, their respective electric fields may interfere with each other. When individual electric fields have different frequencies, interference between such electric fields may result in a region whose electric field has a frequency that is the difference between the frequencies of the individual electric fields. For example, as shown in FIG. 4, one electric field 410 may have a frequency of 2.1 kHz, and another electric field 420 may have a frequency of 2 kHz. Interference between these electric fields may produce a region with an electric field 430 having a frequency of 100 Hz, which is the difference between the frequencies of the individual electric fields 410, 420. Such a region will be referred to herein as an “activation region.” Outside the activation region, however, the individual electric fields 410, 420 may have their respective frequencies.

Current that flows between electrodes is based on the electric field provided by the electrodes. Outside the activation region, currents may have frequencies corresponding to the frequencies of the electric fields through which the currents flow. When such frequencies are greater than 1000 Hz, the currents may not stimulate the tissue they flow through. For target tissue within the activation region, the target tissue may be exposed to current at frequencies less than 1000 Hz, as described in connection with FIG. 4, and at a greater average current than experienced in non-target tissue. When the target tissue includes the muscles of the tongue or the hypoglossal nerve that controls the tongue, the tongue can move. When the target tissue is the genioglossus muscle, the tongue can move forward, away from the airway. In contrast, the non-target tissue may not be exposed to frequencies that cause muscle contraction or nerve stimulation.

The current sufficient to induce stimulation may vary based on the type of target tissue. For example, increasing currents may be provided to stimulate neurons as frequencies increase. In embodiments, stimulating target tissue may also be achieved by modulation of currents, such that different ratios of currents can provide stimulation at varying depths. For example, unilateral stimulation of the various regions, including the motor cortex, may be achieved using a range of ipsilateral: contralateral electrode current ratios between 1:8 through 8:1, for example, 50 uA:12.5 uA. In embodiments, temperature increases may be negligible, such that thermal burns are not a concern.

FIG. 4 is exemplary, and other frequencies, waveforms, and/or numbers of waveforms are contemplated to be within the scope of the present disclosure for the electric fields and currents. For example, in embodiments, the waveforms of the electric fields and currents may be modulated, such as by amplitude modulation. In embodiments, the waveforms of the electric fields and currents may be square waves or other types of pulsed waves, and the pulsed waves may be pulse-width modulated. In embodiments, the waveforms of the electric fields and currents may be modulated in the Fourier domain.

Referring again to FIGS. 1-3, the disclosed systems may apply the approach described in connection with FIG. 4. That is, individual electrical stimulators and electrode pairs may provide electrical output at frequencies that do not stimulate tissue. In the case of neurons, such frequencies may be above 1 kHz. The electrodes may be configured and/or placed at locations such that the combined effect of electric fields provided by the electrodes results in an activation region at target tissue. As described above, current flowing through the activation region may have a frequency that stimulates the target tissue. In the case of neurons, such frequency may be below 1 kHz. In embodiments, in the systems of FIGS. 1-3, electrical stimulators may provide electrical output at frequencies greater than 1 kHz, such as 3000 Hz, 4000 Hz, and/or 10 kHz, among other frequencies, and/or may provide frequencies up to 100 kHz, and/or may provide frequencies up to 1 MHz. The frequency difference of frequencies provided by two different electrode pairs may be between 1 Hz and 1000 Hz, such as between 1 Hz and 100 Hz or between 1 Hz and 200 Hz. The frequency values are exemplary, and other frequency values are contemplated to be within the scope of the present disclosure.

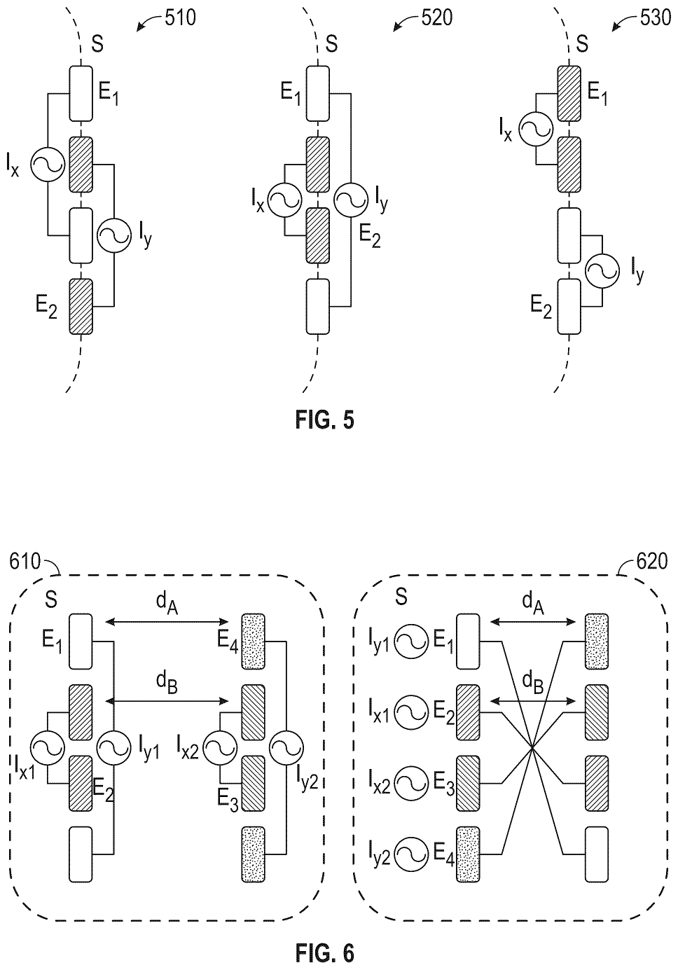

Referring to FIG. 5, various configurations of electrode pairs are shown, including interleaved 510, nested 520, and nearest neighbor 530 electrode configurations. The electrode configurations of FIG. 5 may be used in conjunction with any aspect of the systems, apparatuses, and approaches described in connection with FIGS. 1-4. The electrical stimulators in FIG. 5 (designated by Iₓ and I_{y}) are illustrated to show a separate electrical stimulators for each pair of electrodes. The illustrated electrical stimulators are not intended to show any particular placement or location for the electrical stimulators.

In an “interleaved” electrode configuration 510, a pair of electrodes E1 and a pair of electrodes E2 may be placed on a surface S, such that an electrode from E1 may be between the electrodes of E2 and an electrode of E2 may be between the electrodes of E1. The current paths in the interleaved electrode configuration 510 may intersect in the way shown in FIG. 3; e.g., intersecting at one location. In a “nested” electrode configuration 520, a pair of electrodes E1 and a pair of electrodes E2 may be placed on a surface S, such that the electrodes E2 are between the electrodes of E1 or that the electrodes E1 are between the electrodes of E2. In such a configuration 520, the current paths may intersect in various ways depending on how the electrical outputs of the stimulators are configured; e.g., intersecting at one location or two locations. In a “nearest neighbor” electrode configuration 530, a pair of electrodes E1 and a pair of electrodes E2 may be placed on surface S, such that the electrodes E1 are placed beside the electrodes E2. The currents for the nearest neighbor electrode configuration 530 may intersect in the way shown in FIG. 2; e.g., intersecting at two locations. The illustration is exemplary and variations are contemplated to be within the scope of the present disclosure. In embodiments, there may not be a separate electrical stimulator for each electrode pair.

In embodiments, electrodes may be configured and/or controlled to account for anatomical differences in different people, in order to ensure appropriate treatment of target issue, such as movement of the muscles (e.g., the tongue muscles). For example, without appropriate placement or control of electrodes, the current(s) generated by electrical stimulators may not cause the muscles of a body portion (e.g., the tongue) to contract. In embodiments, an array of electrodes may be incorporated.

FIG. 6 shows exemplary configurations of electrode arrays 610, 620 that include four pairs of electrodes and four electrical stimulators. In embodiments, an electrode array may have a common ground for the electrodes in the array. In embodiments, multiple electrode arrays may have a common ground. In embodiments, an electrode array may have separate grounds in the array. In embodiments, an electrode array may include hardware switches that can be used to selectively activate electrode pairs. In this manner, there is no need to place and move individual electrodes. The electrode configurations of FIG. 6 may be used in conjunction with any aspect of the systems, apparatuses, and approaches described in connection with FIGS. 1-4. In the electrode array 610, electrode pairs E1 and E2 are placed in a nested configuration, and electrode pairs E3 and E4 are placed in a nested configuration. Portions of the two nested configurations may be separated from each other by different distances. For example, a portion of the two nested configurations may be separated by a distance d_{A}, while another portion of the two nested configurations may be separated by a distance d_{B}. In the electrode array 620, the electrodes are placed in the configuration shown in FIG. 3, where a portion of the electrodes may be separated by a distance d_{A}, while another portion of the electrodes may be separated by a distance d_{B}. The illustrated electrical stimulators (Iₓ₁, Iₓ₂, I_{y1}, and I_{y2}) are provided to show that each electrode pair has a separate electrical stimulator and are not intended to show any particular placement or location for the electrical stimulators. The illustration is exemplary and in embodiments, there may not be a separate electrical stimulator for each electrode pair.

In accordance with aspects of the present disclosure, a controller may selectively activate some electrode pairs in the electrode arrays 610, 620 or may activate all electrode pairs in the electrode arrays 610, 620. The ability of the controller to selectively activate some electrode pairs or to activate all electrode pairs, and to adjust electrical characteristics output by the electrode pairs, allows the controller to customize the electrical outputs to a person's anatomy and to treat target tissue in the person in the most effective manner.

In embodiments, in the array of electrodes, more than two pairs of electrodes may be used to create a region of electric field interference, but the region may still have currents with frequencies that do not stimulate tissue (e.g., frequencies above 1000 Hz for neurons). Additional electrical fields of one or more other pairs of electrodes of the array may provide further interference to create an activation region that has currents with frequencies that stimulate tissue (e.g., frequencies below 1000 Hz for neurons). The electrodes may be configured and/or placed such that the activation region occurs in target tissue.

The embodiments of FIGS. 5 and 6 are exemplary. Other electrode configurations and other numbers of electrodes are contemplated to be within the scope of the present disclosure.

FIG. 7 shows an operation for controlling electrical outputs of electrodes. Stimulation may be activated by changing the frequencies of the electrical currents. The operation of FIG. 7 may be applied to any of the systems, apparatuses, and configurations described in connection with FIGS. 1-3, 5, and 7, or with variations of such systems, apparatuses, and configuration. In embodiments, the frequencies of the currents may be changed from a base state, in which the electrode pairs E₁ and E₂ provide currents 710, 720 at the same frequency, to a second state in which a frequency of current between at least one of the electrode pairs is shifted, such that the current in an activation region has a frequency (e.g., the difference between frequencies f₁ and f₂) that provides stimulation of target tissue. When currents 710, 720 are operating at the same frequency, there may be no appreciable stimulation of target tissue. For example, the current 710 between the electrodes E₁ may have a frequency f₁, and the current 720 between the electrodes E₂ may have a frequency f₂. The frequencies f₁ and f₂ may be equal and may be in phase or out of phase. With the detection, onset, or measurement of an “event” 730 (e.g., by a sensor), the frequency f₁ or the frequency f₂ may be shifted to a new frequency f₃. The shift may be performed within a time period t₁. The new frequency f₃ may be held for a time period t₂ and then shifted back to the starting frequency within a time period t₃. With the detection, onset, or measurement of the next “event” 740, the operation may be repeated. Although only two electrode pairs are shown in FIG. 7, the operation may be scaled to apply to more than two pairs of electrodes, such as being applied to the configurations of FIG. 6 or to any other configuration.

Various configurations and operations have been described above. Variations of such configurations and operations are contemplated to be within the scope of the present disclosure. Various additional electrode and electronics configurations may be utilized by the disclosed systems and apparatuses. In embodiments, the electrodes may generate biphasic bipolar pulses, and the electrodes may include an anode and a cathode. In embodiments, the intensity and duty cycle of electrical fields and/or current may be adjustable. In embodiments, the electronics driving the two pairs of electrodes may be separated and electronically isolated to avoid loss of electric field integrity. This may be achieved through solutions such as separate power sources (as shown in FIG. 1) or optically driven circuits. In embodiments, the stimulation current may be driven by optoelectronics, where the generation of electrical currents can be driven by the application of light. In embodiments, the stimulator circuitry may be powered by electromagnetic induction. The disclosed system or apparatus may be powered by an additional power supply, including a battery source. In embodiments, the power supply may be rechargeable or nonchargeable. Such and other variations are contemplated to be within the scope of the present disclosure.

The disclosed configurations and operations may be used to stimulate or otherwise treat various parts of a body. While various uses are contemplated to be within the scope of the present disclosure, the following will describe systems or apparatuses that stimulate tissue to move the tongue to improve airflow. For example, the system or apparatus may stimulate the tongue muscles to increase muscle tone for treating snoring and mild sleep apnea. Unless indicated otherwise, aspects of the descriptions below are intended to be generally applicable to stimulating or treating any tissue.

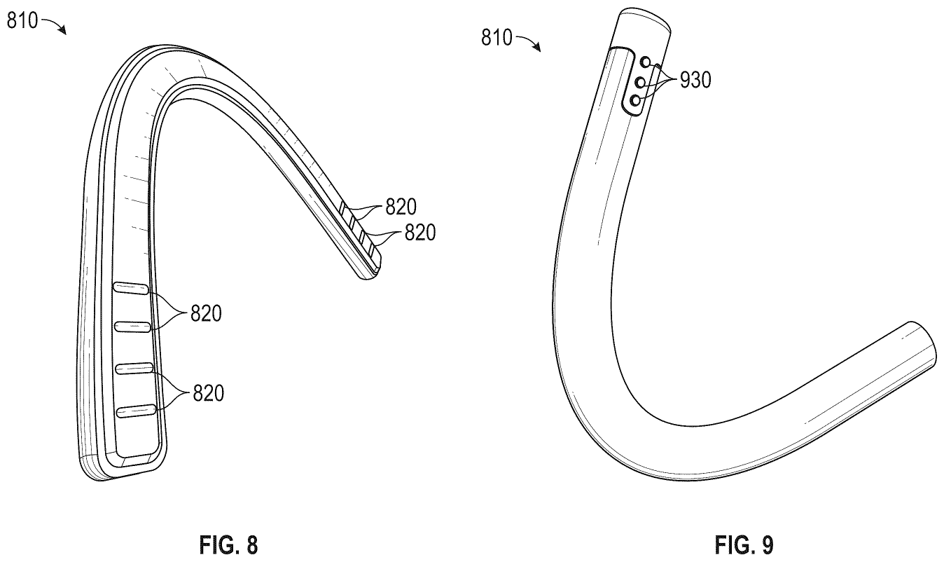

In accordance with aspects of the present disclosure, and as shown in FIGS. 8-11, an exemplary embodiment of an apparatus for electrical stimulation may include a curved housing. In embodiments, the curved housing may have a size and shape that generally follows the curvature of a person's mandible or a person's teeth. In embodiments, the curved housing may be made in different sizes and/or curvatures, and a person may use the particular size and/or curvature that best fits the person. The curved housing has an attachment mechanism that secures the housing to a person, such as adhesives (e.g., tape or glue), straps, and/or magnetics, or other mechanisms for attaching the housing to a person.

FIG. 8 is a diagram of an exemplary curved housing 810. The curved housing 810 includes electrodes 820. The configuration of the electrodes 820 may be any of the configurations described above (e.g., in FIG. 5 or FIG. 6) or may be another configuration. The positions of the electrodes 820 are exemplary, and the electrodes 820 may be located at other positions on the curved housing 810. In embodiments, the curved housing 810 may have a different number of electrodes than that illustrated in FIG. 8. The curvature illustrated in FIG. 8 is exemplary, and other curvatures for the curved housing are contemplated to be within the scope of the present disclosure. As mentioned above, in embodiments, the curved housing may have a size and shape that generally tracks the curvature of a person's mandible, jawline, teeth, or gum line.

In embodiments, some or all of components shown in FIG. 1 may be located within the interior of the curved housing 810, including the batteries shown in FIG. 1. In embodiments, the batteries may be rechargeable. FIG. 9 is a diagram of the curved housing 810 that includes charging pins 930 for charging batteries inside the curved housing 810. The charging pins 930 may be located on a different side of the curved housing 810 from the side that includes the electrodes (820, FIG. 8). The shape of the pins, number of pins, and positions of the pins in FIG. 9 are exemplary, and other shapes, numbers, and positions are contemplated to be within the scope of the present disclosure.

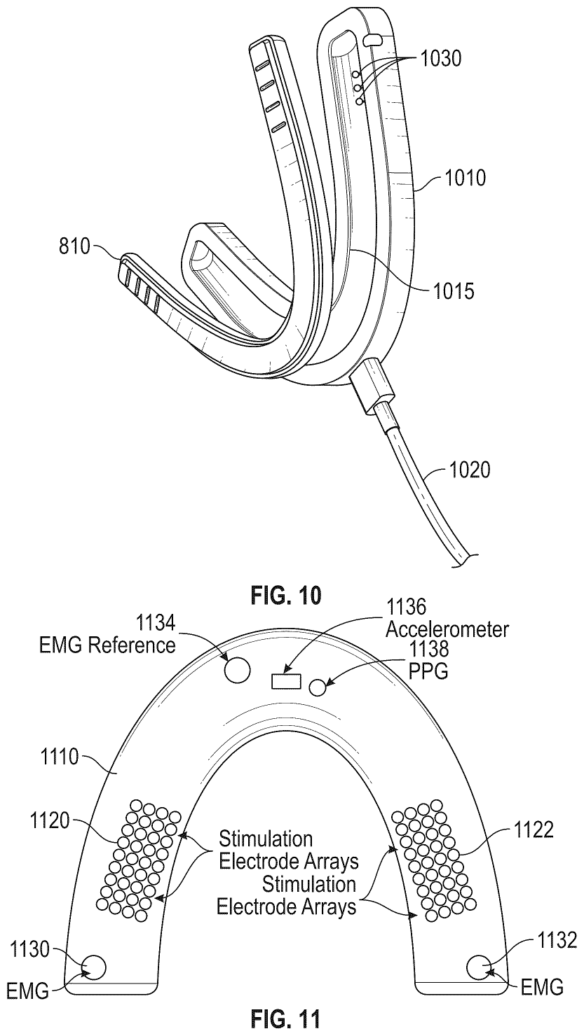

FIG. 10 is a diagram of an exemplary docking device 1010 that receives the curved housing 810 and that charges the batteries inside the curved housing 810. The docking device 1010 includes a recess 1015 that is shaped similarly as the curved housing 810 such that the curved housing 810 may rest within the recess 1015. The docking device 1010 also includes a charging cable that may be plugged into a power port (e.g., wall outlet, USB port, etc.) to convey power for charging the batteries inside the curved housing 810. Power is conveyed to the curved housing 810 by pins 1030 on the docking device 1010. The pins 1030 are positioned to couple with the charging pins 930 on the curved housing 810 when the curved housing 810 is docked on the docking device 1010.

In embodiments, the curved housing 810 and the docking device 1010 may include one or more data pins (not shown) that allow data to be communicated between them. In embodiments, the data pins may be used to update firmware and/or communicate usage data and/or sensor data (e.g., sensor 120, 122, FIG. 1), among other uses. In embodiments, a controller (not shown) in the curved housing 810 may be programmed using a wired data connection that plugs directly to a connector (not shown) in the curved housing 810. The wired data connection may include USB, lightning, micro-USB, or USB-C connections. After programming, the wired data connection hardware may be removed from the curved housing 810. In embodiments, the port-based connector may be designed for ease of removal, such as through pre-scored connections, or connection leads long enough to be easily cut. In embodiments, the port may be designed such that the connection can be re-established, including by reestablishing electrical conductance between leads. In embodiments, the connections may be held together magnetically, allowing a toolless connection and disconnection. In additional embodiments, the connections may be established and stabilized by a physical clip.

FIG. 11 is a diagram of exemplary components of an embodiment having electrode arrays and various sensors. The illustrated components include a curved housing 1110, two electrode arrays 1120, 1122, and various sensors 1130-1138. In embodiments, an electrode array may have a common ground for the electrodes in the array. In embodiments, multiple electrode arrays may have a common ground. In embodiments, an electrode array may have separate grounds in the array. In embodiments, an electrode array may include hardware switches that can be used to selectively activate electrode pairs. In this manner, there is no need to place and move individual electrodes. The electrode arrays 1120, 1122 may be position on the curved housing 1110. In embodiments, some or all of the sensors 1130-1138 may be positioned on the curved housing 1110. In embodiments, some or all of the sensors 1130-1138 may be positioned within the curved housing 1110.

The electrode arrays 1120, 1122 may be configured in the manners described in connection with FIG. 6. For example, certain electrode pairs may be configured such that both electrodes of the pair are in the electrode array 1120 or both electrodes are in the electrode array 1122. Certain electrode pairs may be configured such that one electrode of the pair is in the electrode array 1120 and the other electrode of the pair is in the electrode array 1122. Combinations of such configurations are intended to be within the scope of the present disclosure. In embodiments, a controller (not shown) may selectively activate certain electrode pairs or may activate all of the electrode pairs of the electrode arrays 1120, 1122. As described above, the ability of the controller to activate certain electrode pairs may allow the controller to determine and/or implement a configuration of electrodes that is tailored to the anatomy of the person.

With continuing reference to FIG. 11, the illustrated sensors include electromyography (EMG) sensors 1130, 1132, an EMG reference sensor (1134), an accelerometer 1136, and a PPG sensor 1138. A controller may use sensor data of the EMG sensors 1130-1134 to assess electrical activity in the person resulting from electrical output provided by some or all of the electrodes in the electrode arrays 1120, 1122. Such EMG sensor data may be used to determine a configuration of electrodes from among the electrode arrays 1120, 1125 that would provide effective stimulation or treatment to the person. For example, the apparatus may be applied to a person, and a controller of the apparatus may activate multiple predetermined combinations of electrodes in the electrode arrays 1120, 1122. The controller may assess the data from the EMG sensors 1130, 1132, the accelerometer 1136, and/or the PPG sensor 1138, among other possible sensors. One or more electrode combinations that result in sensor data that satisfy predetermined criteria may be selected by the controller for use with the person.

In embodiments, sensor data of the accelerometer 1136 and/or the PPG sensor 1138 may be used to determine when electrical output from the electrode arrays 1120, 1122 is to be provided. For example, in embodiments, if sensor data from the accelerometer 1136 indicates motion or a sufficient degree of motion, the electrode arrays 1120, 1122 may be activated. As another example, in embodiments, if sensor data from the accelerometer 1136 indicates no motion or a sufficiently-low degree of motion, the electrode arrays 1120, 1122 may be activated in that situation. In embodiments, if sensor data from the PPG sensor 1138 indicates no breath or a sufficiently-low degree of breathing, the electrode arrays 1120, 1122 may be activated in that situation. The accelerometer and PPG sensors 1136, 1138 are merely examples. Other types of sensors and their sensor data may be used to determine when to activate electrodes to provide an electrical output.

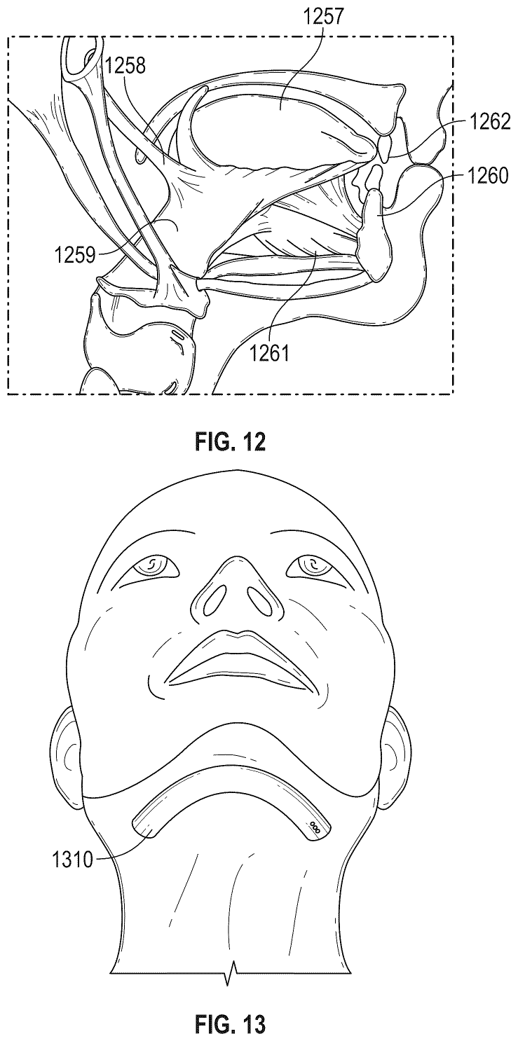

FIG. 12 illustrates various parts of the tongue musculature, including the tongue 1257, the styloglossus 1258, hyoglossus 1259, mandible bone and teeth 1260, and genioglossus 1261. In accordance with aspects of the present disclosure, electrical stimulation may be applied to the tongue musculature using any of the systems, apparatuses, configurations, and operations described above. In embodiments, current(s) may pass through tissue and flow through an activation region in the tongue musculature that is stimulated to cause the tongue muscles and a hypoglossal muscle to move the tongue out of the airway. In embodiments, electrodes may be positioned parallel to the hypoglossal nerve. The electrodes may be configured as shown in any of the configurations of FIG. 5 or FIG. 6, or in other configurations not illustrated herein. In embodiments, a single pair of electrodes may be used to direct a current through the hypoglossal muscle, such as neuromuscular electrical stimulation, causing a contraction when a breath is taken. In embodiments, stimulation of other nerves may assist to open the airway. For example, stimulating the ansa cervicalis innervates sternohyoid, sternothyroid, and/or omohyoid muscles, may improve the airway and thus may be targeted by the apparatus during treatment.

In embodiments, stimulation of other muscles (not shown) of the jaw may be employed to move the jaw forward, opening the airway. These muscles include the lateral pterygoid muscle, innervated by the lateral pterygoid branch of the mandibular nerve, the masseter muscle innervated by the masseteric nerve, and/or a branch of the mandibular nerve and the medial pterygoid muscle, innervated by the medial pterygoid branch of the mandibular nerve.

In embodiments, stimulation may be bilateral or unilateral. In embodiments of bilateral stimulation, each of two sides may be stimulated using different frequencies. For example, in the case of stimulation of the two hypoglossal nerve branches, the use of the same frequencies could result in unintentional phase cancellation, creating a potentially painful stimulation across the jaw.

In accordance with aspects of the present disclosure, causing tongue movement may be beneficial in many procedures. For example, tongue movement may provide an improved airway in patients under the influence or anesthesia or opioids, or during recovery from anesthesia or opioids. In embodiments, tongue movement may provide easier access during intubation, dental procedures, surgery involving the upper airway, or imaging, including endoscopy, implantation of feeding tubes, or gastric surgeries. Any of the systems, apparatuses, and operations disclosed herein may be applied to such procedures and other procedures to cause beneficial tongue movement.

Referring to FIG. 13, and in accordance with aspects of the present disclosure, the anatomy of FIG. 12 may be electrically stimulated by an apparatus 1310 placed on a person under the person's mandible. The apparatus 1310 may include aspects of any of the systems and/or apparatuses described in connection with FIGS. 1-11. For example, the shape of the curved housing may be configured to follow the shape of the person's anatomy (e.g., mandible, jawline, neck, and/or other facial features). In embodiments, the apparatus 1310 may be secured to the person's skin by removable adhesives, or the apparatus 1310 may be secured to the person's head by a strap or a band (not shown), among other mechanisms. The adhesive may contain agents to reduce irritation and redness of the skin, including moisturizers, anti-inflammatory agents, NSAIDs, anesthetics, analgesics, antihistamines, Niacinamide, aloe vera, witch hazel, Sea Buckthorn oil, palmitoleic acid, calendula, chamomile, tiger grass, centella asiatica, hyaluronic acid, vitamin E, squalane, glycerin, and/or cannabidiol. Adhesives, or portions thereof, may contain electroconductive substances, such as salts, and conductive metals like gold, zinc, colloidal silver, copper, and selenium.

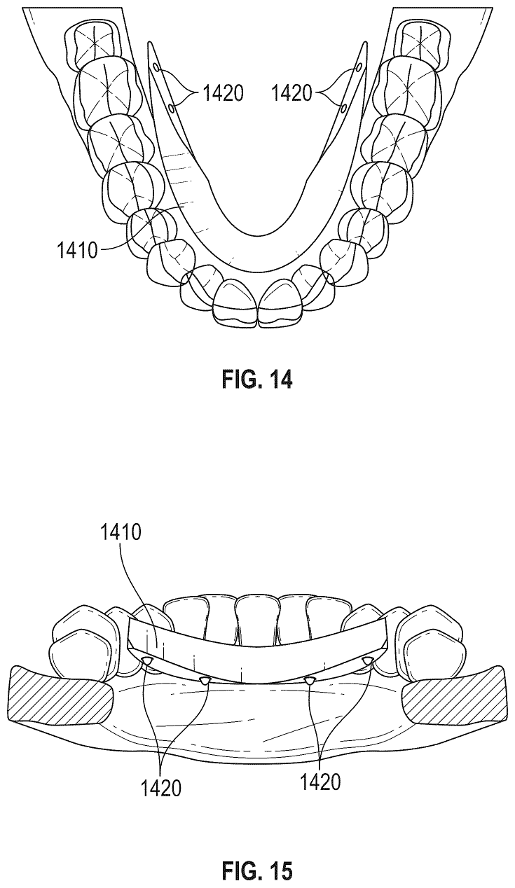

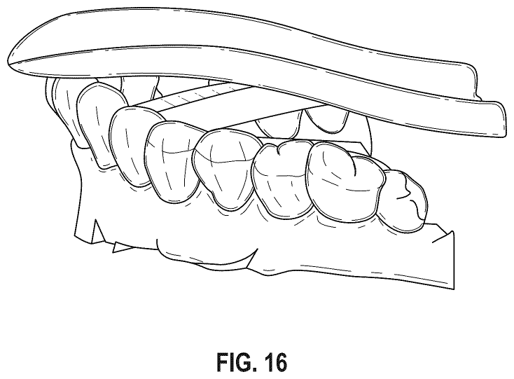

Referring to FIGS. 14 and 15, and in accordance with aspects of the present disclosure, the anatomy of FIG. 12 may be electrically stimulated by an apparatus 1410. The apparatus 1410 includes electrodes 1420 and may be placed within the oral cavity, such as under or above a person's tongue. The apparatus 1410 may include aspects of any of the systems and apparatuses described in connection with FIGS. 1-11. For example, the shape of the curved housing may be configured to follow the shape of the person's anatomy (e.g., teeth, gum line, and/or other facial features). In embodiments, the apparatus 1410 may be substantially flat, but other configurations are contemplated to be within the scope of the present disclosure. The electrodes 1420 may be affixed on the housing and may be positioned in the oral cavity behind the teeth and mandible bone.

The housing of the apparatus 1410 may include a variety of materials. In embodiments, the housing may have a thermoplastic component in contact with a portion of the person's teeth and a rigid acrylic component in contact with a portion of the person's palate. In embodiments, only the thermoplastic component may contact the person's teeth.

In embodiments, the apparatus 1410 may include an orthodontic component (not shown). The orthodontic component may include a dental encasing component having a lingual surface and a labial surface. The dental encasing component may partially or completely overlay the person's teeth and palate. The orthodontic component may also include a rigid component fused to an entire length of the lingual surface, which overlays a portion of the palate of the patient. In embodiments, the orthodontic component may further include a rigid acrylic component fused to a thermoplastic dental encasing component.

The rigid acrylic component may extend from an entire length of the lingual surface of the thermoplastic dental encasing component towards the palate of the patient. The rigid acrylic component may partially or completely cover the person's teeth and palate. In embodiments, only the thermoplastic dental encasing component may fully overlay the person's teeth, and all portions of the palate overlaid by the thermoplastic dental encasing component may also be overlaid by the rigid acrylic component. In embodiments, the rigid acrylic component may have a higher rigidity than the thermoplastic dental encasing component. The rigid acrylic component may also increase a rigidity of an entire arch defined by the thermoplastic dental encasing component without fully overlaying the person's teeth. In embodiments, a cast of the person's lower jaw and palate may be used to create a fitted mouthpiece. The cast may incorporate various components to align the electrodes to contact a base of the mouth.

The apparatus of FIGS. 14 and 15 may be used to electrically stimulate tongue muscles. Without the electrical stimulation, the tongue may position itself closer to the airway and obstruct airflow. Electrical stimulation may cause the tongue muscles to move forward, as shown in FIG. 16, to widen the airway and improve airflow in the person. In embodiments, electrical stimulators may provide electrical output having a frequency up to 100 kHz, such as a frequency around 10 kHz. In various embodiments, the electrical stimulations may provide electrical output having a frequency up to 1 MHz. An activation region having current with frequency between 1 Hz and 100 Hz, or between 1 Hz and 200 Hz, or between 1 Hz and 1000 Hz, may be used to induce involuntary forward tongue flexion. In embodiments, an activation region has a current that has a frequency of 20 Hz, 50 Hz, or 100 Hz. In embodiments, an activation region has a current that has a frequency of 50 Hz. In embodiments, other frequency values may be used. The description above relating to FIGS. 14-16 are exemplary, and variations are contemplated to be within the scope of the present disclosure. For example, the apparatus in the oral cavity may be sized and positioned in other ways not illustrated or described herein.

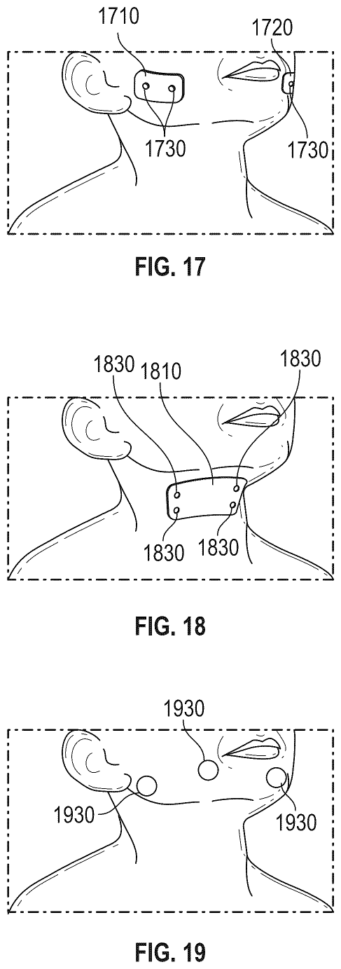

Referring to FIG. 17, an example of electrode patches are shown. The electrode patches 1710, 1720 include electrodes 1730, and the patches 1710, 1720 may be affixed to a person at the positions shown in FIG. 17 (e.g., one patch 1710 on right cheek, and one patch 1720 on left cheek) or at other positions. In embodiments, the electrode patches 1710, 1720 may include removable adhesives that adhere to the person's skin. The electrodes 1730 can be in contact with a person's skin and may be held in position by the adhesives. The adhesive may contain agents to reduce irritation and redness of the skin, such as those describe above in connection with FIG. 13. Adhesives, or portions thereof, may contain electroconductive substances, such as salts, and/or conductive metals like gold, zinc, colloidal silver, copper, and/or selenium. In embodiments, other mechanisms of affixing the electrode patches to the person are contemplated, such as straps or bands.

In embodiments, electrical circuitry (not shown) may be incorporated into the electrode patches 1710, 1720. In embodiments, electrical leads (not shown) may connect the electrodes 1730 of the electrode patches 1710, 1720 to a separate system or apparatus that provides control and electrical output, such as the system of FIG. 1. In embodiments, certain electrical circuitry may be incorporated into the electrode patches 1710, 1720, while certain electrical circuitry may be located in a separate system or apparatus and may be connected to the electrode patches 1710, 1720 by electrical leads.

FIG. 17 is exemplary, and variations are contemplated to be within the scope of the present disclosure. In embodiments, a single electrode patch may be used, as shown in FIG. 18, and the patch may be affixed on a person below the mandible and/or to the person's neck. The single electrode patch 1810 may include multiple electrodes 1830 and may include one or more electrode arrays.

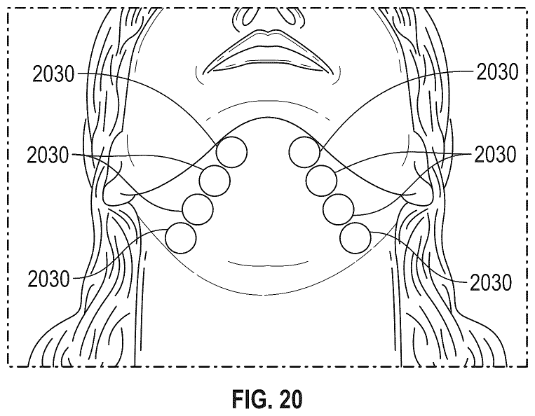

In embodiments, more than two electrode patches may be used, as shown for example, in FIGS. 19 and 20. In embodiments, the patches may be circular patches, such as the circular patches 1930 shown in FIGS. 19 and the circular patches 2030 shown in FIG. 20 and FIGS. 40-42, which may have a diameter of two-inches or a diameter of another size. In embodiments, each circular patch may include a single electrode. As shown in FIGS. 18 and 19, certain patches may be affixed along the left jawline and certain patches may be affixed along the right jawline, and the patches may be located above and/or below the jawline.

Within an electrode patch, or when electrode patches are directly placed on the body, the alignment of electrodes may have different shapes and sizes. For example, electrodes may be arranged in aligned, crossed, nested, nearest neighbor, and/or interleaved arrangements. Some examples of arrangements are shown in FIGS. 5, 6, 20, and 40-42.

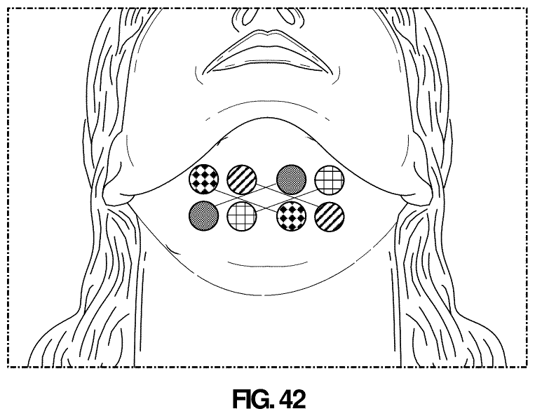

For aligned electrodes arrangements, the electrodes can be paired in a nearest neighbor arrangement (each electrode will be paired with the electrode right next to it; e.g., 1-1′-2-2′ arrangement where 1/1′ are electrodes from pair one and 2/2′ are electrodes from pair 2), interleaved arrangement (electrodes will be paired in a 1-2-1′-2′ arrangement where 1/1′ are electrodes from pair one and 2/2′ are electrodes from pair 2), nested arrangement (one electrode pair will be embedded in the second pair; 1-2-2′-1′ arrangement where 1/1′ are electrodes from pair one and 2/2′ are electrodes from pair 2), or any other aligned arrangements within the scope of the present disclosure, such as those depicted in FIG. 5, FIG. 20 and FIG. 41.

FIG. 40 is an example of bilateral cross electrode arrangements, in which two cross layouts will be placed bilaterally and will not overlap or interact. FIG. 41 is an example of nested electrode arrangements. FIG. 42 is an example of interleaved cross arrangement, in which the cross layouts will be interleaved one with the other and will overlap without interacting. Other electrode arrangements and alignments are within the scope of the present disclosure, such as, without limitation, aligned cross arrangement or separate cross arrangement.

In embodiments, the electrode patch(es) may have shapes and sizes different from those illustrated in FIGS. 17-20 and 40-42. In embodiments, the electrode patch(es) may be placed at various locations, including the locations shown in any of FIGS. 17-20, any of FIGS. 40-42, or any combination of such locations, or at locations different from those illustrated in FIGS. 17-20 and 40-42. For example, the electrode patches may be placed on body portions other than the face or neck, such as on an arm, a leg, the back, or any other portion of a body. Such and other variations are contemplated to be within the scope of the present disclosure.

Accordingly, described above are aspects of systems, apparatuses, and operations for electrically stimulating tissue. In embodiments, any of the systems, apparatuses, and operations disclosed herein may be used while a person is asleep or awake. The components of the systems and apparatuses may contain a variety of materials and may range in durability. In embodiments, the systems and apparatuses may include one or more disposable components and one or more durable, reusable components. In embodiments, the disposable component(s) may include electrodes, sensors, and/or leads for sensors, among other things. In embodiments, the durable component(s) may include electronics, sensors, stimulation circuitry, Bluetooth and wireless communication circuitry, and/or processing circuitry, among other things. For example, the electrodes may be disposable while the electronics are durable and reusable. In embodiments, the electrodes, electronics, and sensors may all be durable, and a removable adhesive sheet may be configured such that the electrodes are in electrical conductance with the skin of the patient. Once used, the removable adhesive sheet may be removed and disposed. In embodiments, the electrodes may also be flexible, such that the electrodes would maintain an electrical connection while remaining comfortable for the patient.

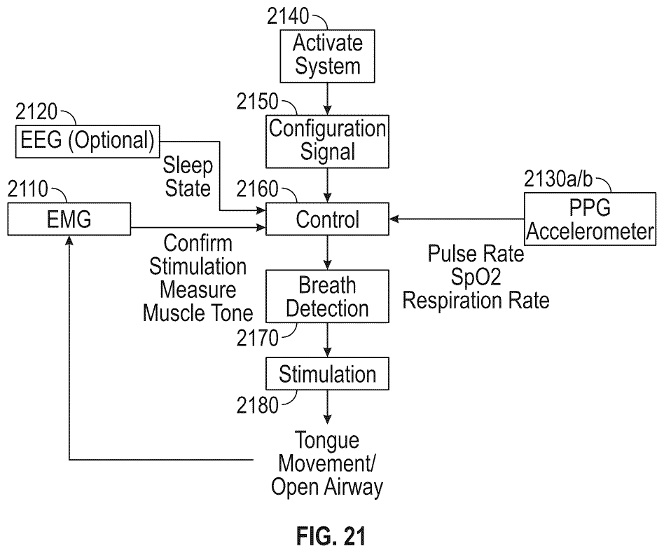

FIG. 21 is a block diagram of exemplary sensors and operations of the systems and apparatuses disclosed herein. Any of the systems and apparatuses disclosed herein may include various sensors for obtaining patient information, including an electromyography (EMG) sensor 2110, an electroencephalogram 2120, a photoplethysmography sensor 2130a, an accelerometer 2130b, and/or a microphone (not shown), among other sensors. In embodiments, the treating physician or medical provider may have access to information provided by the sensors. For example, information provided by the sensors may be downloaded via a docking device, such as the docking device of FIG. 10. In embodiments, for systems and apparatus that include wireless communication capability, information provided by the sensors may be wirelessly uploaded to a central system (not shown). A physician or medical provider may then access such information from the central system.

In embodiments, the central system may be used to generate a compliance report using the information obtained from the sensors. The compliance report may be used to confirm treatment details including actual use, efficacy, sleep quality, and/or the number of apnea or hypopnea events. For example, sleep status, including parameters such as respiration rate, blood oxygen levels, pulse, and/or muscle tone, can be monitored, and stimulation parameters may be adjusted in response (e.g., increase intensity, modify according to breathing pattern).

With continuing reference to FIG. 21, the operations at block 2140 involve activating the disclosed system or apparatus, which may involve powering on and performing startup routines and startup diagnostics. At block 2150, the operations involve generating a confirmation signal that system activation was successful. The operations at block 2160 involve control operations based on sensor data from the sensors 2110-2130a/b, among other possible sensors (not shown) and may be performed by a controller (e.g., 110, FIG. 1). At block 2170, information from the sensors 2110-2130a/b may be used to detect breathing. If information from the sensors indicates that breathing has stopped or meets predetermined criteria, the operation at block 2180 can activate electrodes to stimulate an activation zone and cause the tongue to move and open the airway.

In embodiments, data from one or more EMG sensor(s) 2110 may be used to confirm stimulation and provide additional patient information. For example, a signal corresponding to the stimulation signal may be detectable by the EMG sensor(s) 2110 and may be used by a control operation 2160 to confirm stimulation, electrode placement, or parameter selection. In embodiments, muscle tone may be used as an indicator of sleep stages, such as N1, N2, N3, REM, and Deep Sleep. Information from the EMG sensor 2110 may be used by control operations 2160 to estimate the sleep stage. This data could also be used by control operations 2160 to automatically adjust parameters, such as electrical output parameters of electrical stimulators, among other parameters. In embodiments, a peripheral arterial tone monitor may provide similar patient information. The EMG sensor 2110 may be part of a disposable component, such as a patch that includes the electrodes, or may be part of a durable and reusable component.

In embodiments, data from the electroencephalogram 2120 may be used by control operations 2160 to determine sleep stages, such as N1, N2, N3, REM, and Deep Sleep. These stages may correspond with varying levels of relaxation of the muscles, including general paralysis during REM. This information may be used by control operations 2160 to modify levels of stimulation based on inherent muscle tone during a current stage.

In embodiments, a photoplethysmography sensor 2130a may provide information on pulse rate and blood oxygenation levels. The photoplethysmography sensor 2130a may also include a pulse oximeter or digital holography. Data from this sensor 2130a can also provide an estimate of respiration rate and may be used by control operations 2160 to automatically adjust parameters to improve function of the systems and apparatuses.

In embodiments, an accelerometer 2130b could provide information on respiration rate, movements, and/or sleep position. This data may further be used by control operations 2160 to automatically adjust parameters, such as electrical output parameters of electrical stimulators, among other parameters.

The level of effort required to move the tongue may vary based on sleep position. For example, more effort may be required when sleeping in a supine position. In embodiments, stimulation parameters could change based on sleeping position. In addition, the motion of the mandible may be analyzed to determine apnea events. Data from the EMG 2110, photoplethysmography 2130a, and/or accelerometer 2130b sensors may provide a sleep score, inform a sleep study, and/or assist in a compliance report.

In embodiments, a microphone may be used to detect sounds associated with snoring and breath to calculate a respiration rate. A nasal cannula may also be used to measure airflow and nasal pressure changes. This information may be used to calculate respiration rate and can indicate apnea events.

The control operations 2160 may be configured to adjust stimulation current over time. For example, a lower current may be used during an acclimation period and increased therefrom over the following days or weeks. This may assist with use of the disclosed systems and apparatuses as a transition device. In addition, the stimulation current may be modulated by the control operations 2160 based on the sleep stage. During some sleep stages, especially during REM sleep, muscle tone may decrease more, such that administration of increased current may improve patient outcomes.

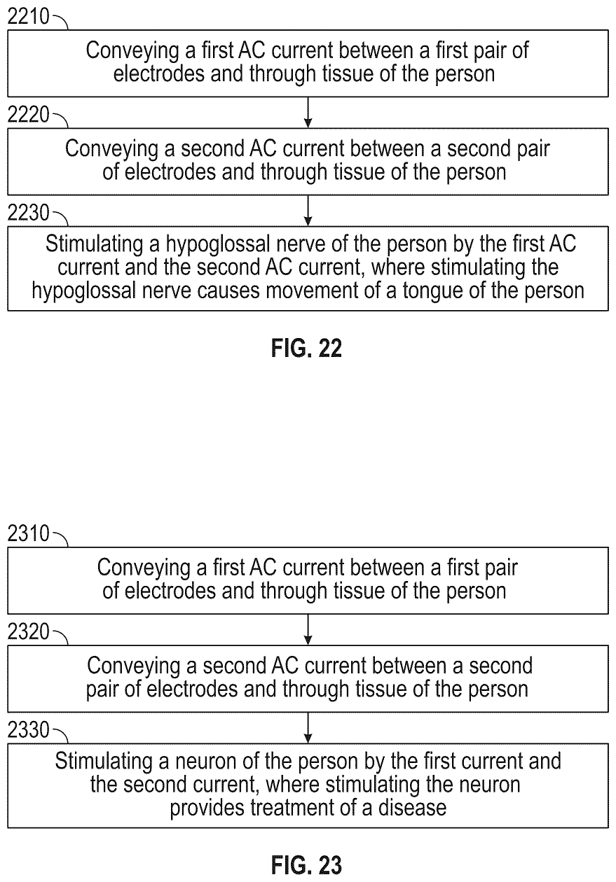

Referring now to FIG. 22, there is shown a flow diagram of an exemplary operation for simulating a hypoglossal nerve. The operation of FIG. 22 may be performed by any of the systems and apparatuses disclosed herein. At block 2210, the operation involves conveying a first AC current between a first pair of electrodes and through tissue of the person. At block 2220, the operation involves conveying a second AC current between a second pair of electrodes and through tissue of the person. At block 2230, the operation involves stimulating a hypoglossal nerve of the person by the first AC current and the second AC current, where stimulating the hypoglossal nerve causes movement of a tongue of the person.

Referring now to FIG. 23, there is shown a flow diagram of an exemplary operation for simulating a neuron. The operation of FIG. 23 may be performed by any of the systems and apparatuses disclosed herein. At block 2310, the operation involves conveying a first AC current between a first pair of electrodes and through tissue of the person. At block 2320, the operation involves conveying a second AC current between a second pair of electrodes and through tissue of the person. At block 2330, the operation involves stimulating a neuron of the person by the first AC current and the second AC current, where stimulating the neuron causes provides treatment of a disease.

In accordance with aspects of the present disclosure, the systems and apparatuses disclosed herein may be used in combination with a host of external devices (not shown). For example, the systems and apparatuses may be in communicative connection with a phone, watch, remote control, and/or tablet. The external devices may store data for various patients. For example, data may be used to find a relationship between the application of electrical stimulation to the patient and the patient's respiratory response to such electrical stimulation, among additional information. The relationship data for large numbers of patients may be aggregated, and thereafter used to identify trends or common components of OSA across various population demographics. The storage device may be a local storage device, or a remote storage device (e.g., accessible via one or more means and/or networks including but not limited to a wide area network (WAN), a wireless local area network (WLAN), a virtual private network (VPN), and the Internet). The data may be made available and manipulated locally or remotely and may be utilized immediately and/or preserved for later utilization and/or apparatuses. In embodiments, a vibrational motor or speaker may be utilized with any of the systems and apparatuses disclosed herein to provide user feedback and alerts or be used as an alarm or notification.

In embodiments, electrical stimulators and sensors of the disclosed systems and apparatuses may communicate via a wired or wireless connection with a mobile device or computer, which collects and analyses data and controls stimulation parameters based on the analyzed data. This computer may be in communication with one or more central systems (e.g., cloud system). These central systems could be in connection with company computers or physician computers. Physicians may provide input through these computers to control stimulation parameters, review sensor data, and generate compliance reports. In embodiments, the mobile device or computer receives patient feedback through an app (e.g., tapping to trigger an accelerometer) or through a button pressed by the patient. For example, the feedback may include data stating that the tongue moved appropriately. In an embodiment, a sensor may also be used to confirm that the tongue or other tissue has moved.

In embodiments, the systems and apparatuses disclosed herein may comprise both a user-worn component (e.g., 1310, FIG. 13) and a desktop component (e.g., 1010, FIG. 10) in wired or wireless communication with one another. The desktop component may be battery-powered or connected to a wall-based main power supply. The desktop component may provide power to a rechargeable battery incorporated with the user-worn component, either wirelessly or through a hardwired connection. In embodiments, the desktop component may include processing hardware.

It is contemplated that the systems, apparatuses, and operations disclosed herein may be used to stimulate or otherwise treat a variety of tissue and/or conditions. For example, lymphatic flow may be modulated by nerve stimulation of the lymph nodes. This restriction leads to a variety of effects, including sequestration of infective bacteria, restriction of colonization by malignant tumor cells, increased production of antibodies in response to a vaccine, and increased class switching to IgG antibodies, such as during allergy desensitization. In an embodiment, lymph nodes and nerves that innervate them may be targeted by electrical stimulation, including the axillary lymph nodes of the armpit and neck, the inguinal and popliteal lymph nodes of the legs, and nerves such as the sciatic nerve.

In embodiments, the systems and apparatuses disclosed herein may be used to target branches of the vagus nerve. Stimulation of the vagus nerve is known to address a variety of diseases and indications. For example, in association with a traumatic bleeding event, or risk thereof, stimulation of the vagus nerve or the splenic nerve may be used as treatment to reduce bleeding time, total blood lost, and risk of bleeding after surgery. In embodiments, stimulation may be applied prophylactically (e.g., before surgery or undergoing activity with a risk of trauma, such as sports or armed forces engagement, or during or after the traumatic event) to address bleeding. When used in association with surgery, stimulation may be applied in the days following surgery to further reduce post-operative bleeding risk. In addition, stimulation of the vagus nerve is known to treat inflammatory diseases, for example, rheumatoid arthritis and inflammatory bowel disease. In embodiments, regular stimulation of the vagus nerve to treat these diseases may be employed. Stimulation of the vagus nerve may further be used to enhance sensory perception and treat migraines, epilepsy, or depression.

The systems and apparatuses disclosed herein may stimulate a variety of other nerves. For example, stimulation of peripheral and spinal sensory nerves can ameliorate pain. Stimulation of the sacral nerve that controls the bladder can control incontinence and overactive bladder syndrome. Regions of the brain may be stimulated, similar to deep brain stimulators, either to target source regions or to halt the spread of a seizure. The phrenic nerve may be stimulated to control the contraction of the diaphragm, allowing the replacement of a mechanical ventilator of improving muscle tone while using a mechanical ventilator. Motor nerves that drive muscular movement may be stimulated to control muscle contraction. For example, in embodiments, the various tissues of the appendages, could be mapped and stimulated by a grid of various electrical fields to control the gross and fine motor movement of the arms, wrists, and hands.

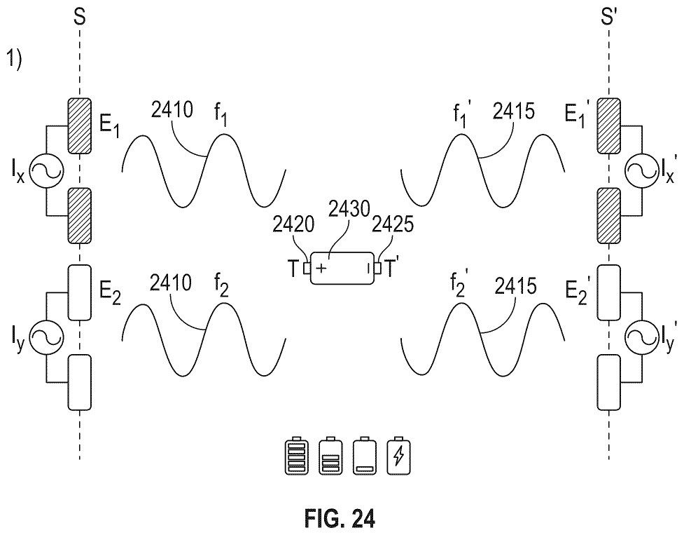

In accordance with aspects of the present disclosure, the systems and apparatuses disclosed herein may be used to provide operational power to an in-vivo battery or in-vivo device, such as an implanted device. As shown in FIG. 24, electrode pairs can be controlled to provide electrical output to a person and to a battery or device 2430 within the person. Certain electrical output 2410 may target one terminal 2420 of a battery or device 2430, and other electrical output 2415 may target another terminal 2425 of the battery or device 2430, thereby creating a differential current between the points 2420, 2425. The periodic signals f1 and f2 2410 from electrodes E1 and E2 can create a potential below a surface S, located at a battery terminal T 2420. Similarly, the periodic signals f1′ and f2′ 2415 from the electrodes E1′ and E2′ can create a potential below a surface S′, located at a battery terminal T′ 2425. The potentials at T 2420 and T′ 2425 may be controlled to allow the battery or device 2430 to enter a charging state. Such charging could be used to power an implanted stimulator 2430 or to charge a battery for a stimulator or other implanted device 2430.

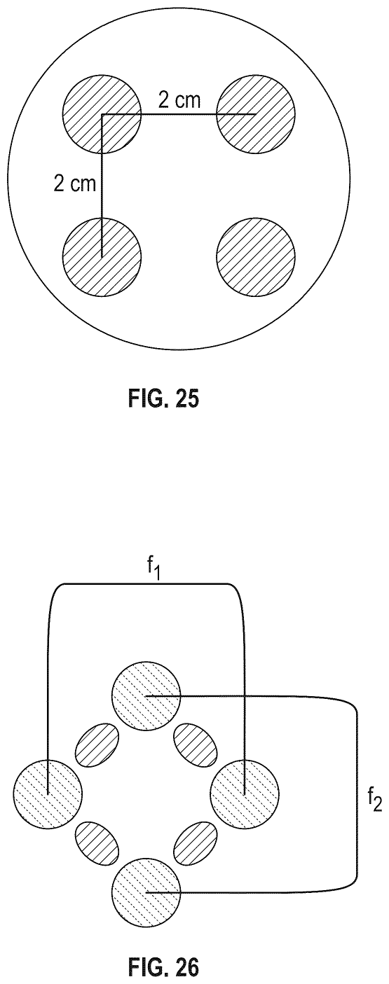

FIGS. 25-39 show further examples, aspects, and embodiments of the present disclosure. In the following description, the term “approximately” means that a property is intended to be achieved, but practical factors in implementing the property may not achieve the intended property exactly. For example, manufacturing factors in implementing physical properties, such as dimensions or shapes, may not exactly achieve the intended dimension or shape due to manufacturing limitations or imperfections.

When activating nerves in patients, e.g., as described in connection with FIG. 2 and FIG. 12, including the hypoglossal nerve, a variety of concerns are weighed. While an activation region has the capability to offer very specific targeting potential, this should be balanced with ease of application by untrained patients and the potential for movement of the nerve relative to skin-adhered electrodes. At times, a slightly larger and less-specific electric field may be a better balance for this type of application.

In various embodiments, electrode placement in a square-shaped configuration is contemplated where electrodes for each separate channel is substantially perpendicular to the other channel. FIG. 25 is a diagram of an exemplary patch with arrangement of four electrodes, in accordance with aspects of the disclosure. The four electrodes are arranged in the shape of a square or approximately in the shape of a square where the four electrodes are positioned at the vertices of the square. In the illustrated embodiment, the edges of the square are 2 cm in length or approximately 2 cm in length. In various embodiments, the edges of the square may have another length.

FIG. 26 is a diagram of exemplary electric fields generated by the electrodes of FIG. 25, in accordance with aspects of the disclosure. The four electrodes include two pairs of electrodes where each pair occupies opposite vertices (i.e., non-adjacent vertices) of the square arrangement. A first voltage frequency f1 is applied to the first pair of electrodes, and a second voltage frequency f2 is applied to the second pair of electrodes. The voltage frequencies may be applied in the manner described in connection with FIGS. 2-4.

As described above in connection with FIG. 4, current that flows between electrodes is based on the electric field provided by the electrodes. Outside the activation region, currents may have frequencies corresponding to the frequencies of the electric fields through which the currents flow. When such frequencies are greater than 1000 Hz, the currents may not stimulate the tissue they flow through. For target tissue within the activation region, the target tissue may be exposed to current at frequencies less than 1000 Hz, as described in connection with FIG. 4, and at a greater average current than experienced in non-target tissue. When the target tissue includes the muscles of the tongue or the hypoglossal nerve that controls the tongue, the tongue can move. When the target tissue is the genioglossus muscle, the tongue can move forward, away from the airway. In contrast, the non-target tissue may not be exposed to frequencies that cause muscle contraction or nerve stimulation.

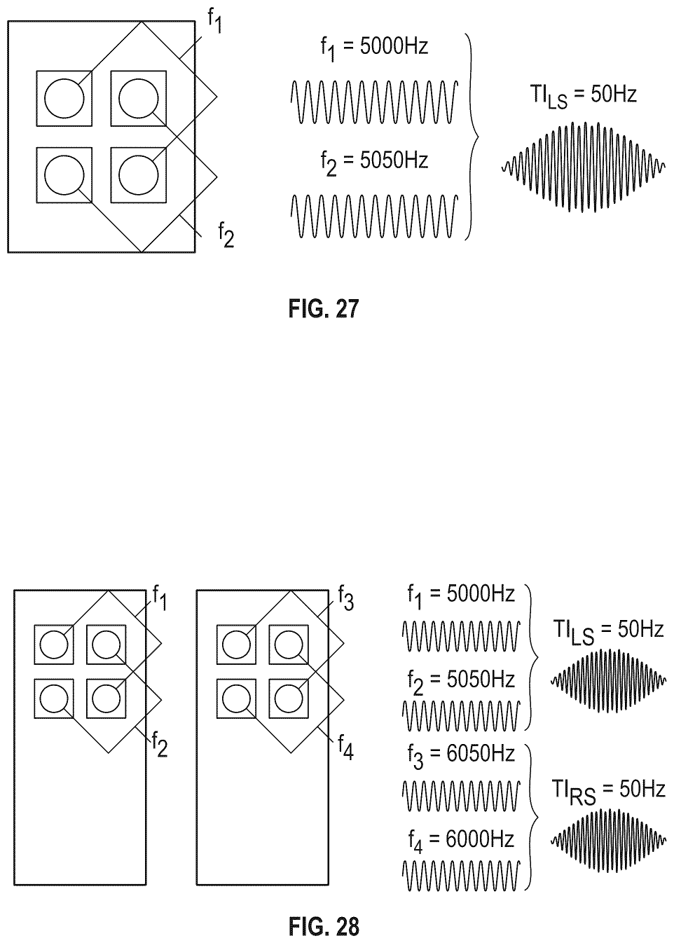

FIG. 27 is a diagram of exemplary voltage frequencies applied to the four electrodes of FIG. 25, in accordance with aspects of the disclosure. In the illustrated embodiment, a voltage frequency of 5000 Hz may be applied to the first pair of electrodes, and a voltage frequency of 5050 Hz may be applied to the second pair of electrodes.

FIG. 28 is a diagram of exemplary voltage frequencies applied to two patches, in accordance with aspects of the disclosure. One patch with four electrodes may be applied to a person's left side, and another patch with four electrodes may be applied to the person's right side. One patch may use the frequencies described in connection with FIG. 27. In the other patch, a voltage frequency of 6000 Hz may be applied to the third pair of electrodes, and a voltage frequency of 6050 Hz may be applied to the fourth pair of electrodes. Due to interference between electric fields, the activation region(s) resulting from the voltages applied to the electrodes may include a current frequency of 50 Hz, in the manner described above herein.

Muscle exhaustion can occur from constant stimulation, but cessation of stimulation could lead to an increase in airway blockage. Similarly, bilateral stimulation improves central movement of the tongue, while unilateral stimulation causes more lateral movement, directionally according to the stimulated side. The improved central movement improves clinical efficacy and modestly improves comfort. Bilateral stimulation also requires less current to be applied, again improving comfort and reducing power requirements.

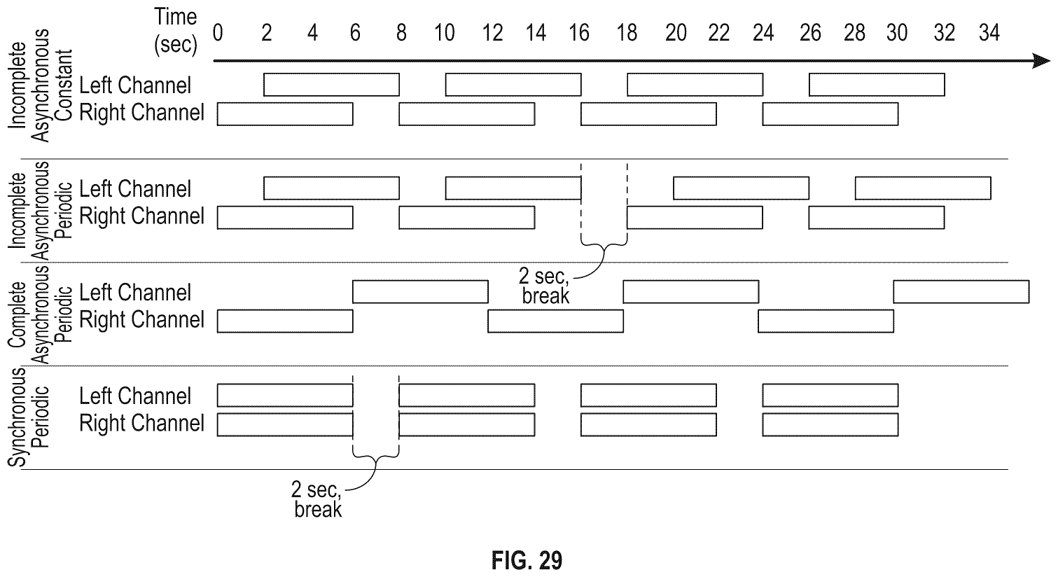

Referring to FIG. 29, bilateral stimulation with pauses reduces tonic muscle activation and therefore improves muscle exhaustion. Muscles recover quickly, so a short pause is generally sufficient. Alternatively, asynchronous bilateral stimulation can be used in several forms. In complete asynchrony, the nerves could be stimulated from side to side, left to right to left, with no overlap. In incomplete asynchrony, during at least some time period, both sides are stimulated at the same time, giving a period of stronger movement or reduced current usage. Periodic breaks in stimulation could be incorporated, or the stimulation could be constant as applied to the body.

In various embodiments, bilateral stimulation is used to stimulate both the left and right hypoglossal nerves, either synchronously or asynchronously. In various embodiments, the current applied during bilateral stimulation is less than that used for unilateral stimulation. In various embodiments, the current applied increases during the night to compensate for nerve desensitization. In various embodiments, the current applied adjusts according to measurements obtained from an electromyography sensor; e.g., if the signal decreases, current applied would increase to increase muscle tone. Similarly, if the EMG signal is too high, current would be decreased.

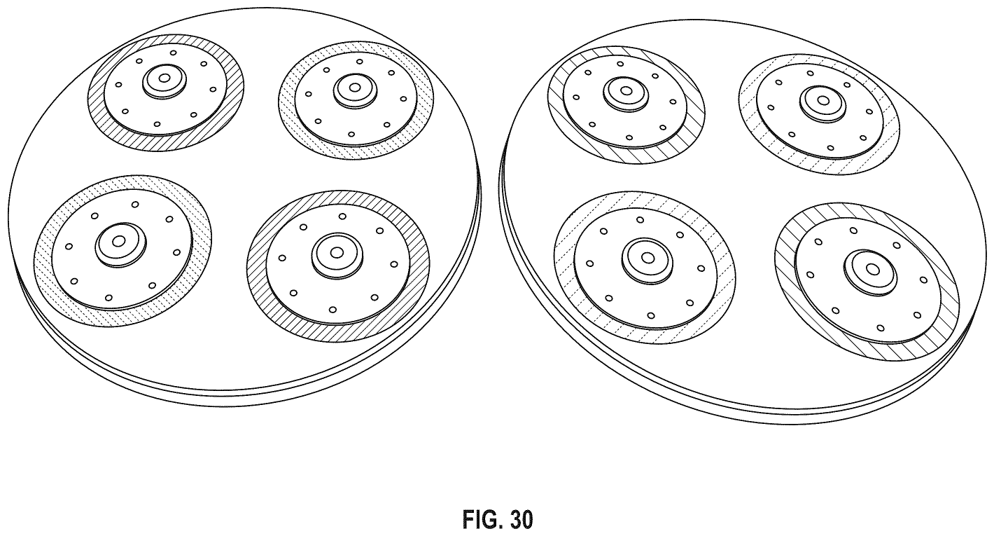

FIG. 30 is a diagram of an exemplary pair of patches, in accordance with aspects of the disclosure. In the illustrated embodiment, the electrodes may be embodied in multielectrode patches. Such patches may be manufactured so that the electrodes are properly configured when adhered to the skin. In various embodiment, the electrodes are arranged in the manner shown in FIG. 25. A printed circuit or wiring could be used to electrically connect one or more electrodes to a connection point. In various embodiments, the lines may be made of silver and the electrodes may be made of carbon. In various embodiments, electrodes may be screen-printed on a thin and flexible polyurethane substrate with a two-step process using Silver (Ag) and Carbon (C) inks.

The electrode connections are in electrical connection with stimulation circuitry. The stimulation circuitry may be housed in an enclosure (e.g., as shown in FIG. 38). The circuitry may be connected by wiring or pad connectors or other ways. The housing may be clipped to a garment, belt, or harness. In various embodiments, the housing may be shaped to rest on the shoulders or be incorporated into a chin strap.

In various embodiments, the patch incorporates a conductive hydrogel. The conductive hydrogel may be surrounded by an insulating layer, including an insulating adhesive. The patch may be adhered to the skin with an acrylic-or silicone-based adhesive. The electrodes may be Ag/AgCl, Ag, or carbon.

In various embodiments, a jig or guide is used to assist in placement of the electrodes. In various embodiments, at least a portion of the jig or guide is removed after electrode placement.

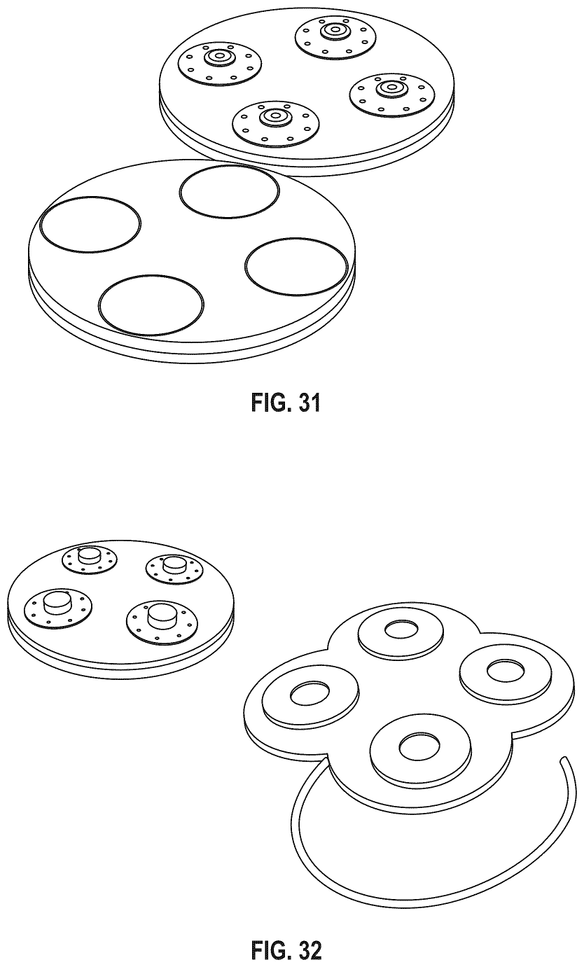

FIG. 31 is a diagram of exemplary patches, with top and bottom surfaces shown, in accordance with aspects of the disclosure. FIG. 32 is a diagram of another exemplary patch with another exemplary connector, in accordance with aspects of the disclosure.

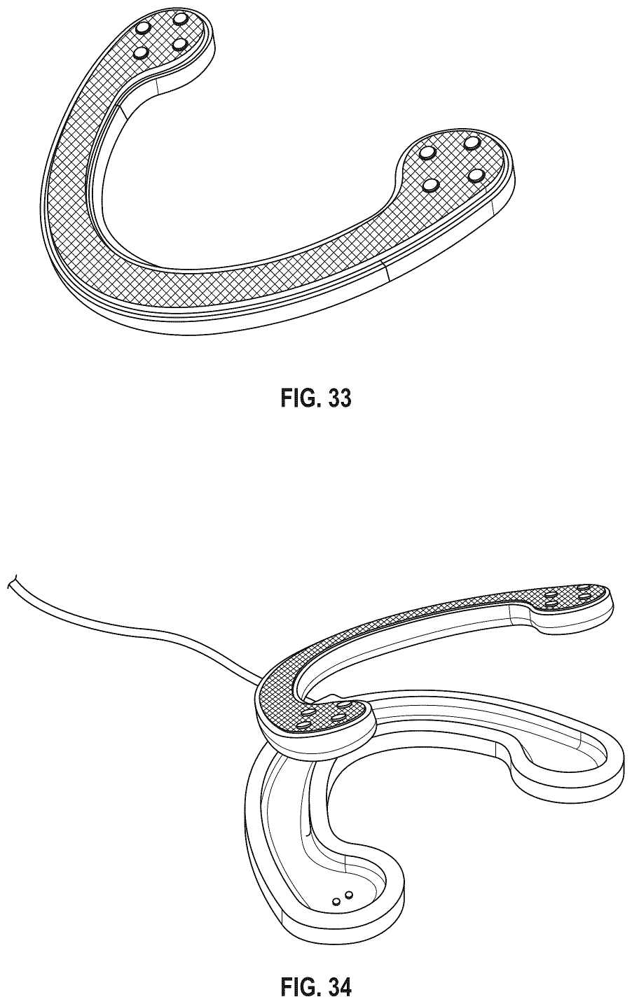

FIG. 33 is a diagram of an exemplary curved housing having four electrodes on each side, in accordance with aspects of the disclosure. The curved housing of FIG. 33 may have various of the features described in connection with FIGS. 8-11 and 13. Any such features are applicable to FIG. 33.

FIG. 34 is a diagram of the curved housing with electrodes of FIG. 33 and an exemplary charger, in accordance with aspects of the disclosure. The charger may have various of the features described in connection with FIG. 10. Any such features are applicable to FIG. 34.

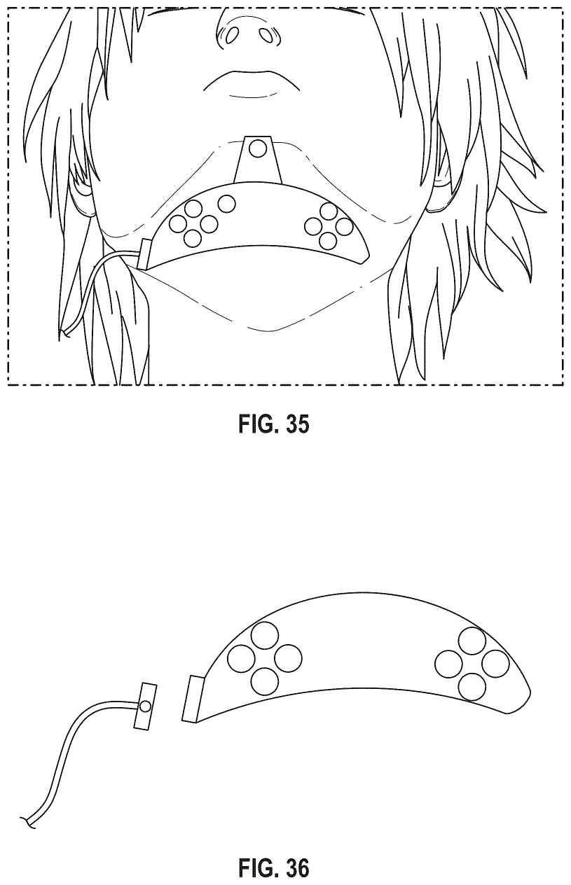

FIG. 35 is a schematic diagram of an exemplary stimulation device with a connection to a cable, in accordance with aspects of the disclosure. In various embodiments, the illustrated stimulation device may be implemented as a patch or as a curved housing.

In various embodiments, the electrodes may be deposited or printed on a substrate characterized by a Young's modulus of less than 30 MPa, e.g., from about 1 MPa to about 30 MPa. An example of a material suitable for use as a substrate includes, without limitation, polyurethane. The diameter of the electrodes can be about 3 mm to about 10 mm. The thickness of the substrate may be about 60 mhi to about 150 mhi, e.g., 80 mhi.

In various embodiments, the patch may be removably connected to wiring or hard-wired. Wiring may be connected by a harness, snaps, magnetic pegs, Zero Insertion Force (ZIF) clips, or other means. The connector may establish electrical connection with one or more electrodes.

In various embodiments, the stimulation device (e.g., patch or curved housing) may incorporate sensors including, without limitation, an accelerometer, a gyroscope, a photoplethysmogram (PPG), electroencephalography (EEG), electrocardiogra Electrooculography (EOG) (recording eye movement), electro-olfactography (EOLG), and/or electromyography (EMG).

In various embodiments, one or more sensors may be incorporated into the wiring or the cable connector, as shown in FIG. 36. FIG. 36 is a schematic diagram of another exemplary stimulation device with a sensor on the cable or the cable connector, in accordance with aspects of the disclosure. The sensor(s) may including an accelerometer, a gyroscope, a photoplethysmogram (PPG), electroencephalography (EEG), electrocardiogra Electrooculography (EOG) (recording eye movement), electro-olfactography (EOLG), and/or electromyography (EMG).

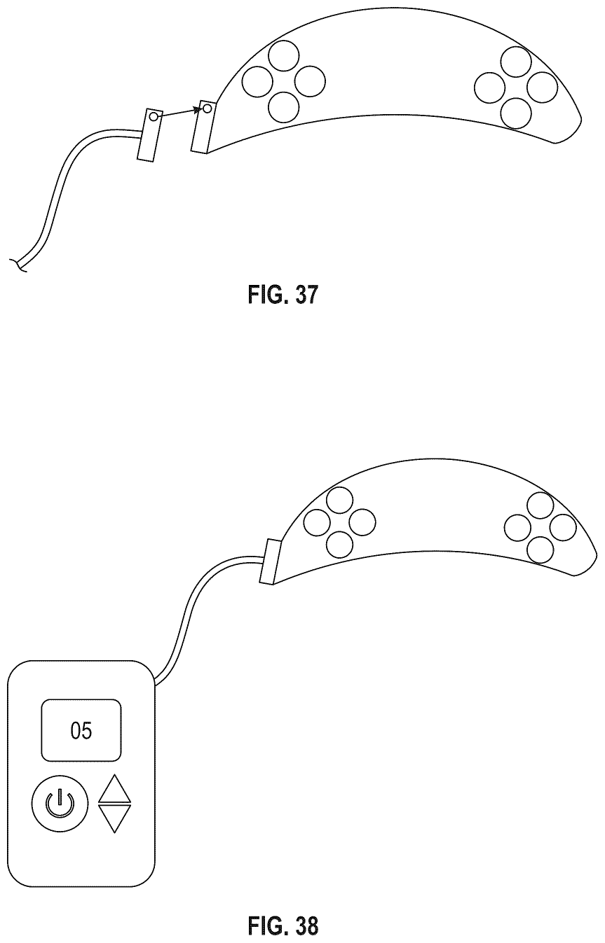

FIG. 37 is a schematic diagram of an exemplary stimulation device with a magnetic connection to a cable, in accordance with aspects of the disclosure. In the illustrated embodiment, the connector portion of the wire incorporates a magnetic portion that aids in alignment and connection with the stimulation device (e.g., patch or curved housing). A physical clip may optionally further secure the connection.

FIG. 38 is a schematic diagram of an exemplary stimulation system having a stimulation device and a control device connected by a cable, in accordance with aspects of the disclosure. The control device may include various of the features described in connection with FIGS. 1, 2, and 21. Any such features are applicable to FIG. 38.

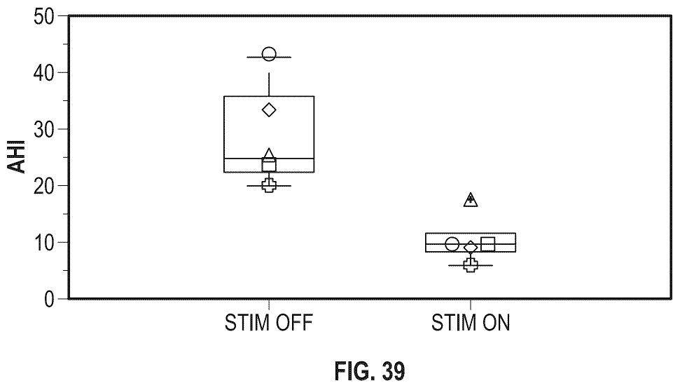

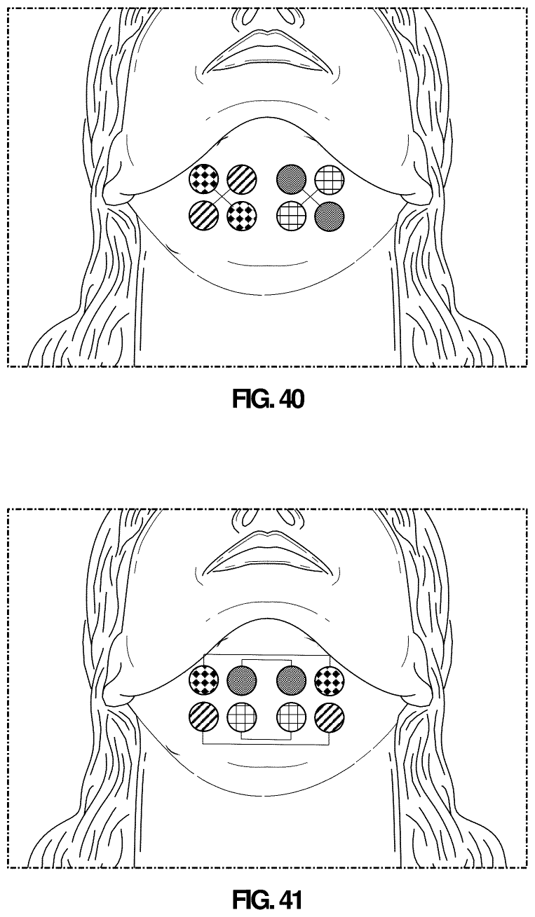

FIG. 39 is a graph showing clinical Apnea Hypopnea Index (AHI) data for patients when stimulated and when not stimulated by a stimulation device according to he present disclosure. As shown by the clinical data of FIG. 39 and as described below, the stimulation device of the present disclosure may be used to meaningfully affect sleep diseases.

During sleep studies in humans, obstructive sleep apnea (OSA) is characterized using sleep summaries and more precisely Apnea Hypopnea Index (AHI). According to the American Academy of Sleep Medicine (AASM), apneas are identified as ≥90% flow reduction events lasting more than 10 seconds; hypopneas are set as a ≥30% flow reduction for more than 10 seconds with a decrease of O₂ saturation ≥3% or arousal. This way, apneas and hypopneas can be identified, and AHI will be characterized as the number of apnea events plus the number of hypopnea over the total sleep time (AHI=(#apnea+#hypopnea)/TST). Patients are then diagnosed as healthy patients (AHI<5), mild OSA patients (5<AHI<15), moderate OSA patients (15<AHI<30), or severe OSA patients (AHI>30).

In an experimental study of the disclosed technology, twelve patients were recruited and treated overnight during an in-clinic polysomnography. Upon retrospective review, it was discovered that one patient significantly exceeded the targeted BMI range of <35 (BMI=49.8), and therefore this patient's data were excluded from analysis. The primary endpoint was % responders (AHI reduction >50% and an AHI below 20), with an expected 50% response rate. Among all patients completing the study, 45% of patients ( 5/11) responded to the stimulation therapy with a high gender-dependent effect on therapy outcome ( 4/4 women versus 1/7 men). Among patients that responded to dTI stimulation, their AHI decreased by 61.62%±18.99% (AHI=28.85±9.23 when the stimulation is off against 10.38±4.23 when the stimulation is on, p-value_{Wilcoxon test}<0.001). When looking at the amplitude threshold necessary to elicit tongue movement/tension, there was a clear difference—women needed less current to evoke the same behavior compared to men (4.6±1.34 mA for women versus 7.86±1.46 mA for men). Overall, dTI stimulation led to a significant decrease in AHI for a subset of OSA patients with a clear improvement in the severity of sleep apnea.

Participants: For enrollment in this study, patients were aged 18 years or older, in general good health, and diagnosed with moderate or severe sleep apnea based on an in-sleep laboratory polysomnogram (PSG) within 6 months in a sleep diagnostics facility with an AHI of 15 to 50/hour, and agree to be alcohol or caffeine free on the day of the study. A total of 12 patients were included in this early feasibility study (7 men and 5 women), aged 48.1±10.7 years old and with an average BMI of 30.6±6.8 kg/m2.

Exclusion criteria: Patients were excluded from this early feasibility study if they are diagnosed with no, mild, or too severe OSA (AHI>50). In addition, patients with a BMI under 18.5 Kg/m2 and over 32 Kg/m2 will be excluded from the present study. Finally, patients must not be pregnant or have one or more of the following diseases: enlarged tonsils (size 3-4) and/or adenoids, nasal polyps, neuromuscular disease, hypoglossal nerve palsy, abnormal pulmonary function tests, severe pulmonary hypertension, valvular heart disease, heart failure (New York Heart Association, NYHA III-IV), recent myocardial infarction, significant cardiac arrhythmias, atrial fibrillation, stroke, opioid usage, uncontrolled hypertension, known allergic reaction to adhesives or latex, active psychiatric disease, co-existing non-respiratory sleep disorder, or significant metal implants including pacemakers.

Study design: This early feasibility study was designed as an open-label, monocentric, single-group treatment trial where patients were under their own control.

Qualified patients underwent a night PSG wearing the stimulation device for bilateral hypoglossal nerve stimulation. The device consists of 4 pairs of commercially available gel-based electrodes, 2 pairs for each HN placed on a 2 cm square around the digastric anterior belly muscle and oriented to be aligned with the HN. The 8 electrodes in total were placed at the bedside by the technician once the patient is ready to go to bed. Stimulation was applied through the pairs of electrodes as described above herein. Before going to sleep, the patients were subjected to an amplitude titration (from 0.1 to 8 mA) to evaluate what stimulation amplitude is needed to elicit a tongue movement; when the movement threshold was reached, the stimulation was turned off and the patient could go to sleep. Once the patient was asleep, the stimulation was switched on at the lowest amplitude (1 mA) and ramped up by steps of 0.5 mA until the pre-defined threshold is attained. Any time during the night, the patient could ask to pause the treatment and be disconnected from the stimulators, to use the bathroom for example. Lastly, the patients were given a questionnaire at the end of the night to relate their experience with the stimulation device.

Outcomes measures: The primary outcome of the study aims to evaluate the efficiency of the stimulation device through the measurement of AHI during the in-laboratory investigation. A positive endpoint is defined as a decrease of the AHI of at least 50% with an overnight AHI of 20 or less. The secondary outcomes included Percentage Sleep Time at SaO2 <90% (Determined by the time below a SaO2 level of 90% compared to that at baseline) and Safety based on a description of all reported adverse events. The tertiary outcome will focus on exploring patients' comfort by wearing the stimulation device overnight using retrospective questionaries.

Statistical analysis: For the primary outcome, sleep summaries were analyzed in a single-blinded approach under Rule 1A of the AAAS guidelines (apneas are identified as ≥90% flow reduction events lasting more than 10 seconds; hypopneas are set as a ≥30% flow reduction for more than 10 seconds with a decrease of 02 saturation ≥3%), and the sleep technician who analyzed the sleep summaries was not aware of the stimulation status. The obtained AHIs were compared to the ones obtained during the baseline (initial PSG established within 6 months before the present study). To determine whether the therapy was efficient or not, Wilcoxon tests were realized with α=1% or by Student's T-test, two-tailed and paired. As shown by the clinical data of FIG. 39 and as described below, the stimulation device of the present disclosure may be used to meaningfully affect sleep diseases.

Further examples of the present disclosure are described below.

Example 1. An apparatus for electrical stimulation of tissue, comprising:
a first pair of electrodes configured to contact a person and to convey a first AC current between the first pair of electrodes through tissue of the person; and
a second pair of electrodes configured to contact the person and to convey a second AC current between the second pair of electrodes and through tissue of the person,
wherein the first pair of electrodes and the second pair of electrodes are configured to be positioned on the person such that the first AC current and the second AC current are simultaneously conveyed through a target tissue of the person, the first AC current and the second AC current configured to stimulate the target tissue.

Example 2. The apparatus of Example 1, wherein the first AC current has a first frequency and the second AC current has a second frequency, wherein the first frequency and the second frequency are both greater than 1000 Hz.

Example 3. The apparatus of Example 2, wherein current conveyed through the target tissue has a frequency different from the first frequency and different from the second frequency.

Example 4. The apparatus of Example 3, wherein the current conveyed through the target tissue has a frequency equivalent to a frequency difference between the first frequency and the second frequency.

Example 5. The apparatus of Example 4, wherein the frequency difference is less than 1000 Hz.

Example 6. The apparatus of Example 1, wherein the first pair of electrodes and the second pair of electrodes are in one of: an interleaved configuration, a nested configuration, or a nearest neighbor configuration.

Example 7. The apparatus of Example 1, further comprising:
a first battery configured to supply power to the first pair of electrodes; and a second battery configured to supply power to the second pair of electrodes,
wherein the first battery does not supply power to the second pair of electrodes, and wherein the second battery does not supply power to the first pair of electrodes.

Example 8. The apparatus of Example 1, further comprising a controller configured to set parameters of the first AC current and the second AC current, the parameters including the first frequency of the first AC current and the second frequency of the second AC current.

Example 9. The apparatus of Example 8, wherein the controller is configured to set the parameters of the first AC current and the second AC current by:
setting the first frequency and the second frequency to a base frequency value;
in response to a determination that an event has occurred, setting the first frequency to a different frequency value that is different from the base frequency value within a first time period after the event;
maintaining the first frequency at the different frequency value and the second frequency at the base frequency value for a second time period after the first time period; and
reverting the first frequency to the base frequency value within a third time period after the second time period.

Example 10. The apparatus of Example 8, further comprising a first electrode array and a second electrode array,
wherein the first electrode array includes the first pair of electrodes, and wherein the second electrode array includes the second pair of electrodes.

Example 11. The apparatus of Example 10, wherein the controller is further configured to: determine a subset of the first electrode array and a subset of the second electrode array that, when activated, causes stimulation of the target tissue;
activate the subset of the first electrode array without activating all electrodes of the first electrode array, wherein the subset of the first electrode array includes the first pair of electrodes; and
activate the subset of the second electrode array without activating all electrodes of the second electrode array, wherein the subset of the second electrode array includes the second pair of electrodes.

Example 12. The apparatus of Example 8, further comprising an electromyography (EMG) sensor configured to provide sensor data,
wherein the controller is further configured to determine, based on the EMG sensor data, at least one of: muscle tone or confirmation of tissue stimulation, and
wherein the controller sets the parameters of the first AC current and the second AC current based on at least one of: the muscle tone or the confirmation of tissue stimulation.

Example 13. The apparatus of Example 8, further comprising an electroencephalogram (EEG) sensor configured to provide sensor data,
wherein the controller is further configured to determine, based on the EEG sensor data, a sleep state of the person, and
wherein the controller sets the parameters of the first AC current and the second AC current based on the sleep state of the person.

Example 14. The apparatus of Example 8, further comprising a photoplethysmography (PPG) sensor configured to provide sensor data,
wherein the controller is further configured to determine, based on the PPG sensor data, respiration rate of the person, and
wherein the controller sets the parameters of the first AC current and the second AC current based on the respiration rate.

Example 15. The apparatus of Example 8, further comprising a housing,
wherein the controller is located within an interior of the housing, and
wherein the first pair of electrodes and the second pair of electrodes are located at a surface of the housing.

Example 16. The apparatus of Example 1, further comprising:
at least one patch configured to adhere to skin of the person, the at least one patch comprising the first pair of electrodes and the second pair of electrodes;
at least one battery; and
at least one electrical connection configured to electrically couple the at least one battery with the at least one patch.

Example 17. The apparatus of Example 1, wherein tissue in at least a portion of a path of the first AC current is not stimulated by the first AC current, and
wherein tissue in at least a portion of a path of the second AC current is not stimulated by the first AC current.

Example 18. The apparatus of Example 1, further comprising:
a third pair of electrodes configured to contact the person and to convey a third AC current between the third pair of electrodes and through tissue of the person; and
a fourth pair of electrodes configured to contact the person and to convey a fourth AC current between the fourth pair of electrodes and through tissue of the person,
wherein the first pair of electrodes, the second pair of electrodes, the third pair of electrodes, and the fourth pair of electrodes are configured to be positioned on the person such that the first AC current, the second AC current, the third AC current, and the fourth AC current are simultaneously conveyed through the target tissue of the person and stimulate the target tissue.

Example 19. The apparatus of Example 18, wherein tissue in at least a portion of a path of the third AC current is not stimulated by the third AC current, and
wherein tissue in at least a portion of a path of the fourth AC current is not stimulated by the fourth AC current.

Example 20. The apparatus of Example 1, wherein the target tissue comprises a hypoglossal nerve of the person.

Example 21. A method for electrical stimulation of tissue by a first pair of electrodes in contact with a person and a second pair of electrodes in contact with the person, the method comprising:
simultaneously:
conveying a first AC current between the first pair of electrodes and through tissue of the person, and
conveying a second AC current between the second pair of electrodes and through tissue of the person,
wherein the first AC current and the second AC current are simultaneously conveyed through a target tissue of the person based on positioning of the first pair of electrodes and the second pair of electrodes on the person, the first AC current and the second AC current configured to stimulate the target tissue.

Example 22. The method of Example 21, wherein the first AC current has a first frequency and the second AC current has a second frequency, wherein the first frequency and the second frequency are both greater than 1000 Hz.

Example 23. The method of Example 22, wherein current conveyed through the target tissue has a frequency different from the first frequency and different from the second frequency.

Example 24. The method of Example 23, wherein the current conveyed through the target tissue has a frequency equivalent to a frequency difference between the first frequency and the second frequency.

Example 25. The method of Example 24, wherein the frequency difference is less than 1000 Hz.

Example 26. The method of Example 21, further comprising:
setting parameters of the first AC current and the second AC current, the parameters including the first frequency of the first AC current and the second frequency of the second AC current.

Example 27. The method of Example 26, wherein setting the parameters of the first AC current and the second AC current comprises:
setting the first frequency and the second frequency to a base frequency value;
in response to a determination that an event has occurred, setting the first frequency to a different frequency value that is different from the base frequency value within a first time period after the event;
maintaining the first frequency at the different frequency value and the second frequency at the base frequency value for a second time period after the first time period; and
reverting the first frequency to the base frequency value within a third time period after the second time period.

Example 28. The method of Example 26, wherein the first pair of electrodes is in a first electrode array, and
wherein the second pair of electrodes is in a second electrode array.

Example 29. The method of Example 28, further comprising:
determining a subset of the first electrode array and a subset of the second electrode array that, when activated, causes stimulation of the target tissue;
activating the subset of the first electrode array without activating all electrodes of the first electrode array, wherein the subset of the first electrode array includes the first pair of electrodes; and
activating the subset of the second electrode array without activating all electrodes of the second electrode array, wherein the subset of the second electrode array includes the second pair of electrodes.

Example 30. The method of Example 26, wherein setting the parameters of the first AC current and the second AC current comprises:
determining, based on sensor data from an electromyography (EMG) sensor, at least one of: muscle tone or confirmation of tissue stimulation; and
setting the parameters of the first AC current and the second AC current based on at least one of: the muscle tone or the confirmation of tissue stimulation.

Example 31. The method of Example 26, wherein setting the parameters of the first AC current and the second AC current comprises:
determining, based sensor data from an electroencephalogram (EEG) sensor, a sleep state of the person; and
setting the parameters of the first AC current and the second AC current based on the sleep state of the person.

Example 32. The method of Example 26, wherein setting the parameters of the first AC current and the second AC current comprises:
determining, based on the sensor data from a photoplethysmography (PPG) sensor, respiration rate of the person; and
setting the parameters of the first AC current and the second AC current based on the respiration rate.

Example 33. The method of Example 21, wherein tissue in at least a portion of a path of the first AC current is not stimulated by the first AC current, and
wherein tissue in at least a portion of a path of the second AC current is not stimulated by the first AC current.

Example 34. The method of Example 21, further comprising: simultaneously:
conveying a third AC current between a third pair of electrodes and through tissue of the person, and
conveying a fourth AC current between a fourth pair of electrodes and through tissue of the person, and
wherein the first AC current, the second AC current, the third AC current, and the fourth AC current are simultaneously conveyed through the target tissue based on positioning of the third pair of electrodes and the fourth pair of electrodes on the person.

Example 35. The method of Example 34, wherein tissue in at least a portion of a path of the third AC current is not stimulated by the third AC current, and
wherein tissue in at least a portion of a path of the fourth AC current is not stimulated by the fourth AC current.

Example 36. The method of Example 21, wherein the target tissue comprises a hypoglossal nerve of the person.

Example 37. An apparatus for electrical stimulation of tissue, comprising: a housing configured to be affixed to tissue of a person;
a first battery and a second battery located within the housing;
a first pair of electrodes electrically coupled with the first battery;
a second pair of electrodes electrically coupled with the second battery; and
an attachment mechanism configured to affix at least one of the housing or the first and second pairs of electrodes to tissue of a person such that the first and second pairs of electrodes contact tissue of the person,
wherein the first battery does not supply power to the second pair of electrodes, and wherein the second battery does not supply power to the first pair of electrodes.

Example 38. The apparatus of Example 37, wherein the first pair of electrodes and the second pair of electrodes are located at a surface of the housing.

Example 39. The apparatus of Example 37, further comprising:
at least one patch configured to adhere to skin of the person, the at least one patch comprising the first pair of electrodes and the second pair of electrodes; and
at least one electrical connection configured to couple the first battery and the second battery with the at least one patch.

Example 40. The apparatus of Example 37, wherein electrodes are placed in aligned or separate cross arrangement.

Example 41. The apparatus of Example 37, wherein electrodes pairs are stimulating in nearest neighbor, interleaved, nested, bilateral cross, interleaved cross configuration or any other configuration possible using the apparatus of Example 37.

Example 42. The apparatus of Example 39, wherein the at least one patch comprises a first patch and a second patch,
wherein the first patch comprises the first pair of electrodes, and wherein the second patch comprises the second pair of electrodes.

Example 43. The apparatus of Example 39, wherein the at least one patch comprises: a first patch comprising a first electrode of the first pair of electrodes;
a second patch comprising a second electrode of the first pair of electrodes;
a third patch comprising a first electrode of the second pair of electrodes; and a fourth patch comprising a second electrode of the second pair of electrodes.

Example 44. The apparatus of Example 37, wherein the housing is configured to be placed within and removable from an oral cavity of a person.

Example 45. The apparatus of Example 42, wherein the housing has a shape that tracks a gum line of the oral cavity of the person.

Example 46. The apparatus of Example 37, wherein the housing is configured to be affixed to skin of the person under a mandible of the person.

Example 47. The apparatus of Example 44, wherein the housing has a shape that tracks a jawline of the person.

Example 48. The apparatus of Example 37, further comprising charging pins located at a surface of the housing, the charging pins electrically coupled with the first battery and the second battery,
wherein the charging pins are configured to convey power to recharge the first battery and the second battery.

Example 49. The apparatus of Example 37, further comprising a wireless communication device located within the housing, the wireless communication device configured to provide wirelessly communication capability to communicate with a central system.

Example 50. The apparatus of Example 47, further comprising a controller located within the housing, wherein the wireless communication device is configured to communicate a firmware update to the controller, the firmware update provided by the central system.

Example 51. The apparatus of Example 47, further comprising at least one sensor configured to provide sensor data, wherein the wireless communication device is configured to communicate the sensor data for delivery to the central system.

Example 52. A method for electrical stimulation of a hypoglossal nerve, comprising:
conveying a first AC current between a first pair of electrodes and through tissue of the person;
conveying a second AC current between a second pair of electrodes and through tissue of the person; and
stimulating a hypoglossal nerve of the person by the first AC current and the second AC current, wherein stimulating the hypoglossal nerve causes movement of a tongue of the person.

Example 53. The method of Example 50, wherein the first AC current has a first frequency and the second AC current has a second frequency, wherein the first frequency and the second frequency are both greater than 1000 Hz.

Example 54. The method of Example 51, wherein current conveyed through the hypoglossal nerve has a frequency different from the first frequency and different from the second frequency.

Example 55. The method of Example 52, wherein the current conveyed through the hypoglossal nerve has a frequency equivalent to a frequency difference between the first frequency and the second frequency.

Example 56. The method of Example 53, wherein the frequency difference is less than 1000 Hz.

Example 57. A method for electrical stimulation of neurons to treat a disease, comprising: conveying a first AC current between a first pair of electrodes and through tissue of a person;
conveying a second AC current between a second pair of electrodes and through tissue of the person; and
stimulating a neuron of the person by the first AC current and the second AC current, wherein stimulating the neuron provides treatment of a disease.

Example 58. The method of Example 55, wherein the first AC current has a first frequency and the second AC current has a second frequency.

Example 59. The method of Example 56, wherein current conveyed through the neuron has a frequency different from the first frequency and different from the second frequency.

Example 60. The method of Example 57, wherein the current conveyed through the neuron has a frequency equivalent to a frequency difference between the first frequency and the second frequency.

Example 61. An apparatus for electrical stimulation of tissue, comprising:
a first pair of electrodes configured to contact a person and to convey a first AC current between said first pair of electrodes through tissue of said person; and
a second pair of electrodes configured to contact said person and to convey a second AC current between said second pair of electrodes and through tissue of said person,
wherein said first pair of electrodes and said second pair of electrodes are configured to be positioned on said person such that said first AC current and said second AC current are simultaneously conveyed through a target tissue of said person,
wherein said first AC current and said second AC current are configured to stimulate the target tissue,
wherein the first AC current has a first frequency and the second AC current has a second frequency,
wherein the first frequency and the second frequency are both greater than 1000 Hz,
wherein the difference in the first frequency and the second frequency is between 1 and 200 Hz,
wherein the electrodes do not touch the target tissue,
wherein said target tissue is a hypoglossal nerve,
and wherein conveyance of said first AC current and said second AC current causes the genioglossus muscle to contract.

Example 62. The apparatus of Example 61, wherein said target tissue is the left and right hypoglossal nerve.

Example 63. The apparatus of claim 62, further comprising:
a third pair of electrodes configured to contact a person and to convey a third AC current between said third pair of electrodes through tissue of said person; and
a fourth pair of electrodes configured to contact said person and to convey a fourth AC current between said fourth pair of electrodes and through tissue of said person,
wherein said third AC current and said fourth AC current are configured to stimulate the target tissue,
wherein the third AC current has a third frequency and the fourth AC current has a fourth frequency,
wherein the third frequency and the fourth frequency are both greater than 1000 Hz,
wherein the difference in the third frequency and the fourth frequency is between 1 and 200 Hz, and
wherein the electrodes do not touch the target tissue.

Example 64. The apparatus of Example 63,
wherein said first AC current and said second AC current substantially stimulate the left hypoglossal nerve and said third AC current and said fourth AC current substantially stimulate the right hypoglossal nerve.

Example 65. The apparatus of Example 64, further comprising:
a controller configured to set parameters of the first AC current and the second AC current, the parameters including the first frequency of the first AC current and the second frequency of the second AC current,
wherein the controller is configured to set the parameters of the first AC current and the second AC current by:
setting the first frequency and the second frequency to a base frequency value;
in response to a determination that an event has occurred, setting the first frequency to
a different frequency value that is different from the base frequency value within a first time period after the event;
maintaining the first frequency at the different frequency value and the second frequency at the base frequency value for a second time period after the first time period; and
reverting the first frequency to the base frequency value within a third time period after the second time period,
further comprising a controller configured to set parameters of the third AC current and the fourth AC current, the parameters including the third frequency of the third AC current and the fourth frequency of the fourth AC current,
wherein the controller is configured to set the parameters of the third AC current and the fourth AC current by:
setting the third frequency and the fourth frequency to a base frequency value; in response to a determination that an event has occurred, setting the third frequency to a different frequency value that is different from the base frequency value within a first time period after the event;
maintaining the third frequency at the different frequency value and the fourth frequency at the base frequency value for a second time period after the first time period; and
reverting the third frequency to the base frequency value within a third time period after the second time period.

Example 66. The apparatus of Example 65, further comprising:
a housing,
a first battery and a second battery located within the housing;
said first pair of electrodes is electrically coupled with the first battery;
said second pair of electrodes is electrically coupled with the second battery; and
an attachment mechanism configured to affix at least one of the housing or the first and second pairs of electrodes to tissue of a person such that the first and second pairs of electrodes contact tissue of the person,
wherein the first battery does not supply power to the second pair of electrodes, and
wherein the second battery does not supply power to the first pair of electrodes.

Example 67. The apparatus of Example 66,
wherein said first battery does not supply power to the third pair of electrodes, and
wherein the second battery does not supply power to the fourth pair of electrodes.

Example 68. A method for electrical stimulation of tissue by a first pair of electrodes configured to contact a person and to convey a first AC current between said first pair of electrodes through tissue of said person and a second pair of electrodes configured to contact said person and to convey a second AC current between said second pair of electrodes and through tissue of said person,
said method comprising:
simultaneously:
conveying a first AC current between the first pair of electrodes and through tissue of the person, and
conveying a second AC current between the second pair of electrodes and through tissue of the person,
wherein said first AC current and said second AC current are configured to stimulate the target tissue,
wherein the first AC current has a first frequency and the second AC current has a second frequency,
wherein the first frequency and the second frequency are both greater than 1000 Hz,
wherein the difference in the first frequency and the second frequency is between 1 and 200 Hz,
wherein the electrodes do not touch the target tissue,
wherein said target tissue is a hypoglossal nerve,
and wherein conveyance of said first AC current and said second AC current causes the genioglossus muscle to contract.

Example 69. The method of Example 68, wherein said target tissue is the left and right hypoglossal nerve.

Example 70. The method of Example 69, further utilizing a third pair of electrodes configured to contact a person and to convey a third AC current between said third pair of electrodes through tissue of said person; and
a fourth pair of electrodes configured to contact said person and to convey a fourth AC current between said fourth pair of electrodes and through tissue of said person,
said method further comprising:
simultaneously:
conveying a first AC current between the first pair of electrodes and through tissue of the person, and
conveying a second AC current between the second pair of electrodes and through tissue of the person,
wherein said third AC current and said fourth AC current are configured to stimulate the target tissue,
wherein the third AC current has a third frequency and the fourth AC current has a fourth frequency,
wherein the third frequency and the fourth frequency are both greater than 1000 Hz,
wherein the difference in the third frequency and the fourth frequency is between 1 and 200 Hz, and
wherein the electrodes do not touch the target tissue.

Example 71. The method of Example 70,
wherein said first AC current and said second AC current substantially stimulate the left hypoglossal nerve and said third AC current and said fourth AC current substantially stimulate the right hypoglossal nerve.

Example 72. The method of Example 71, further comprising a controller configured to set parameters of the first AC current and the second AC current, the parameters including the first frequency of the first AC current and the second frequency of the second AC current,
said method further comprising:
setting the first frequency and the second frequency to a base frequency value;
in response to a determination that an event has occurred, setting the first frequency to a different frequency value that is different from the base frequency value within a first time period after the event;
maintaining the first frequency at the different frequency value and the second frequency at the base frequency value for a second time period after the first time period; and
reverting the first frequency to the base frequency value within a third time period after the second time period,
further comprising a controller configured to set parameters of the third AC current and the fourth AC current, the parameters including the third frequency of the third AC current and the fourth frequency of the fourth AC current,
wherein the controller is configured to set the parameters of the third AC current and the fourth AC current by:
setting the third frequency and the fourth frequency to a base frequency value; in response to a determination that an event has occurred, setting the third frequency to a different frequency value that is different from the base frequency value within a first time period after the event;
maintaining the third frequency at the different frequency value and the fourth frequency at the base frequency value for a second time period after the first time period; and
reverting the third frequency to the base frequency value within a third time period after the second time period.

Example 73. The method of Example 72, further comprising
a housing,
a first battery and a second battery located within the housing;
said first pair of electrodes is electrically coupled with the first battery;
said second pair of electrodes is electrically coupled with the second battery; and
an attachment mechanism configured to affix at least one of the housing or the first and second pairs of electrodes to tissue of a person such that the first and second pairs of electrodes contact tissue of the person,
wherein the first battery does not supply power to the second pair of electrodes, and
wherein the second battery does not supply power to the first pair of electrodes.

Example 74. The method of Example 73,
wherein said first battery does not supply power to the third pair of electrodes, and
wherein the second battery does not supply power to the fourth pair of electrodes.

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the present disclosure and may be embodied in various forms. For instance, although certain aspects herein are described as separate aspects, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an aspect,” “in aspects,” “in various aspects,” “in some aspects,” or “in other aspects” may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Is understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The aspects described with reference to the figures are presented only to demonstrate certain examples of the present disclosure. Other aspects, clements, steps, methods, and techniques that are insubstantially different from those described above and/or in the appended claims are also intended to be within the scope of the present disclosure.

What is claimed is:

1. An apparatus for electrical stimulation of tissue, comprising:
four electrodes comprising:
a first pair of electrodes configured to contact a person and to convey a first AC current between the first pair of electrodes through tissue of the person, and
a second pair of electrodes configured to contact the person and to convey a second AC current between the second pair of electrodes and through tissue of the person,
wherein the four electrodes are positioned approximately in a shape of a square, wherein the first pair of electrodes and the second pair of electrodes are each positioned at opposite vertices of the square, and
wherein the first pair of electrodes and the second pair of electrodes are configured to be positioned on the person such that the first AC current and the second AC current are simultaneously conveyed through a target tissue of the person, the first AC current and the second AC current configured to stimulate the target tissue.

2. The apparatus of claim 1, wherein the first AC current has a first frequency and the second AC current has a second frequency, wherein the first frequency and the second frequency are both at least 5000 Hz.

3. The apparatus of claim 2, wherein current conveyed through the target tissue has a frequency different from the first frequency and different from the second frequency.

4. The apparatus of claim 3, wherein the current conveyed through the target tissue has a frequency equivalent to a frequency difference between the first frequency and the second frequency.

5. The apparatus of claim 4, wherein the frequency difference is approximately 50 Hz.

6. The apparatus of claim 2, wherein the first frequency is approximately 5000 Hz and the second frequency is approximately 5050 Hz.

7. The apparatus of claim 2, wherein the first frequency is approximately 6000 Hz and the second frequency is approximately 6050 Hz.

8. The apparatus of claim 1, wherein edges of the square are approximately 2 cm.

9. The apparatus of claim 1, further comprising a patch configured to adhere to skin of the person, the patch comprising the first pair of electrodes and the second pair of electrodes.

10. The apparatus of claim 1, further comprising:
a second set of four electrodes comprising:
a third pair of electrodes configured to contact the person and to convey a third AC current between the third pair of electrodes and through tissue of the person, and
a fourth pair of electrodes configured to contact the person and to convey a fourth AC current between the fourth pair of electrodes and through tissue of the person,
wherein the four electrodes of the second set are positioned approximately in a shape of a second square, wherein the third pair of electrodes and the fourth pair of electrodes are each positioned at opposite vertices of the second square.

11. The apparatus of claim 10, wherein the first AC current has a first frequency, the second AC current has a second frequency, the third AC current has a third frequency, and the fourth AC current has a fourth frequency, wherein the third frequency and the fourth frequency are both at least 5000 Hz.

12. The apparatus of claim 11, wherein the first frequency is approximately 5000 Hz, the second frequency is approximately 5050 Hz, third frequency is approximately 6000 Hz, and the fourth frequency is approximately 6050 Hz.

13. The apparatus of claim 10, wherein edges of the second square are approximately 2 cm.

14. The apparatus of claim 10, wherein the third pair of electrodes and the fourth pair of electrodes are configured to be positioned on the person such that the third AC current and the fourth AC current are simultaneously conveyed through the target tissue of the person, the third AC current and the fourth AC current configured to stimulate the target tissue.

15. The apparatus of claim 14, wherein the target tissue comprises a hypoglossal nerve of the person.

16. The apparatus of claim 10, further comprising a patch configured to adhere to skin of the person, the patch comprising the first pair of electrodes, the second pair of electrodes, the third pair of electrodes, and the fourth pair of electrodes.

17. The apparatus of claim 16, wherein the patch is configured to be affixed to skin of the person under a mandible of the person.

18. The apparatus of claim 17, wherein the patch has a shape that tracks a jawline of the person.

19. The apparatus of claim 1, wherein the four electrodes are arranged in one of: an aligned arrangement or a cross arrangement.

20. The apparatus of claim 10, wherein at least some of the first pair of electrodes, the second pair of electrodes, the third pair for electrodes, and the fourth pair of electrodes are arranged in one of: a bilateral cross arrangement, or an interleaved cross arrangement.

21. An apparatus for electrical stimulation of tissue, comprising:
a first pair of electrodes configured to contact a person and to convey a first AC current between said first pair of electrodes through tissue of said person; and
a second pair of electrodes configured to contact said person and to convey a second AC current between said second pair of electrodes and through tissue of said person,
wherein said first pair of electrodes and said second pair of electrodes are configured to be positioned on said person such that said first AC current and said second AC current are simultaneously conveyed through a target tissue of said person,
wherein said first AC current and said second AC current are configured to stimulate the target tissue,
wherein the first AC current has a first frequency and the second AC current has a second frequency,
wherein the first frequency and the second frequency are both greater than 1000 Hz,
wherein a difference in the first frequency and the second frequency is between 1 and 200 Hz,
wherein the first pair of electrodes and the second pair of electrodes do not touch the target tissue,
wherein said target tissue is a hypoglossal nerve, and
wherein conveyance of said first AC current and said second AC current causes a genioglossus muscle to contract.

22. The apparatus of claim 21, wherein said target tissue includes a left hypoglossal nerve and a right hypoglossal nerve.

23. The apparatus of claim 22, further comprising:
a third pair of electrodes configured to contact a person and to convey a third AC current between said third pair of electrodes through tissue of said person; and
a fourth pair of electrodes configured to contact said person and to convey a fourth AC current between said fourth pair of electrodes and through tissue of said person,
wherein said third AC current and said fourth AC current are configured to stimulate the target tissue,
wherein the third AC current has a third frequency and the fourth AC current has a fourth frequency,
wherein the third frequency and the fourth frequency are both greater than 1000 Hz,
wherein a difference in the third frequency and the fourth frequency is between 1 and 200 Hz, and
wherein the third pair of electrodes and the fourth pair of electrodes do not touch the target tissue.

24. The apparatus of claim 23, wherein said first AC current and said second AC current substantially stimulate the left hypoglossal nerve and said third AC current and said fourth AC current substantially stimulate the right hypoglossal nerve.

25. The apparatus of claim 24, further comprising:
a first controller configured to set parameters of the first AC current and the second AC current, the parameters including the first frequency of the first AC current and the second frequency of the second AC current, wherein the first controller is configured to set the parameters of the first AC current and the second AC current by:
setting the first frequency and the second frequency to a base frequency value;
in response to a determination that an event has occurred, setting the first frequency to a different frequency value that is different from the base frequency value within a first time period after the event;
maintaining the first frequency at the different frequency value and the second frequency at the base frequency value for a second time period after the first time period; and
reverting the first frequency to the base frequency value within a third time period after the second time period,
a second controller configured to set parameters of the third AC current and the fourth AC current, the parameters including the third frequency of the third AC current and the fourth frequency of the fourth AC current, wherein the controller is configured to set the parameters of the third AC current and the fourth AC current by:
setting the third frequency and the fourth frequency to a base frequency value;
in response to a determination that an event has occurred, setting the third frequency to a different frequency value that is different from the base frequency value within a first time period after the event;
maintaining the third frequency at the different frequency value and the fourth frequency at the base frequency value for a second time period after the first time period; and
reverting the third frequency to the base frequency value within a third time period after the second time period.

26. The apparatus of claim 25, further comprising:
a housing;
a first battery and a second battery located within the housing;
said first pair of electrodes is electrically coupled with the first battery;
said second pair of electrodes is electrically coupled with the second battery; and
an attachment mechanism configured to affix at least one of the housing or the first and second pairs of electrodes to tissue of a person such that the first and second pairs of electrodes contact tissue of the person,
wherein the first battery does not supply power to the second pair of electrodes, and
wherein the second battery does not supply power to the first pair of electrodes.

27. The apparatus of claim 26,
wherein said first battery does not supply power to the third pair of electrodes, and
wherein the second battery does not supply power to the fourth pair of electrodes.

28. A method for electrical stimulation of tissue by a first pair of electrodes configured to contact a person and to convey a first AC current between said first pair of electrodes through tissue of said person and a second pair of electrodes configured to contact said person and to convey a second AC current between said second pair of electrodes and through tissue of said person, the method comprising:
simultaneously:
conveying a first AC current between the first pair of electrodes and through tissue of the person, and
conveying a second AC current between the second pair of electrodes and through tissue of the person,
wherein said first AC current and said second AC current are configured to stimulate a target tissue,
wherein the first AC current has a first frequency and the second AC current has a second frequency,
wherein the first frequency and the second frequency are both greater than 1000 Hz,
wherein a difference in the first frequency and the second frequency is between 1 and 200 Hz,
wherein the first pair of electrodes and the second pair of electrodes do not touch the target tissue,
wherein said target tissue is a hypoglossal nerve, and
wherein conveyance of said first AC current and said second AC current causes a genioglossus muscle to contract.

29. The method of claim 28, wherein said target tissue includes a left hypoglossal nerve and a right hypoglossal nerve.

30. The method of claim 29, further comprising:
utilizing a third pair of electrodes configured to contact a person and to convey a third AC current between said third pair of electrodes through tissue of said person;
a fourth pair of electrodes configured to contact said person and to convey a fourth AC current between said fourth pair of electrodes and through tissue of said person; and
simultaneously:
conveying a third AC current between the third pair of electrodes and through tissue of the person, and
conveying a fourth AC current between the fourth pair of electrodes and through tissue of the person,
wherein said third AC current and said fourth AC current are configured to stimulate the target tissue,
wherein the third AC current has a third frequency and the fourth AC current has a fourth frequency,
wherein the third frequency and the fourth frequency are both greater than 1000 Hz,
wherein a difference in the third frequency and the fourth frequency is between 1 and 200 Hz, and
wherein the electrodes do not touch the target tissue.

31. The method of claim 30,
wherein said first AC current and said second AC current substantially stimulate the left hypoglossal nerve and said third AC current and said fourth AC current substantially stimulate the right hypoglossal nerve.

32. The method of claim 31, further comprising:
setting parameters of the first AC current and the second AC current, the parameters including the first frequency of the first AC current and the second frequency of the second AC current;
setting the first frequency and the second frequency to a base frequency value;
in response to a determination that an event has occurred, setting the first frequency to a different frequency value that is different from the base frequency value within a first time period after the event;
maintaining the first frequency at the different frequency value and the second frequency at the base frequency value for a second time period after the first time period;
reverting the first frequency to the base frequency value within a third time period after the second time period;
setting parameters of the third AC current and the fourth AC current, the parameters including the third frequency of the third AC current and the fourth frequency of the fourth AC current;
setting the third frequency and the fourth frequency to a base frequency value;
in response to a determination that an event has occurred, setting the third frequency to a different frequency value that is different from the base frequency value within a first time period after the event;
maintaining the third frequency at the different frequency value and the fourth frequency at the base frequency value for a second time period after the first time period; and
reverting the third frequency to the base frequency value within a third time period after the second time period.
